(19) United States
(12) Patent Application Publication
(10) Pub. No.: US 2026/0053432 A1
(43) Pub. Date: Feb. 26, 2026

(54) POSITIONAL SLEEP THERAPY USING EAR-WORN DEVICES

(71) Applicant: ResMed Pty Ltd., Bella Vista (AU)

(72) Inventors: Liam Holley, Bella Vista (AU); Nicholas James MEALEY, Fort Lee, NJ (US)

(73) Assignee: ResMed Pty Ltd., Bella Vista (AU)

(21) Appl. No.: 19/304,787

(22) Filed: Aug. 20, 2025

Related U.S. Application Data
(60) Provisional application No. 63/685,325, filed on Aug. 21, 2024.
(60) Provisional application No. 63/685,324, filed on Aug. 21, 2024.
(60) Provisional application No. 63/685,322, filed on Aug. 21, 2024.
(60) Provisional application No. 63/813,243, filed on May 28, 2025.
(60) Provisional application No. 63/813,238, filed on May 28, 2025.
(60) Provisional application No. 63/813,222, filed on May 28, 2025.

(51) Int. Cl. A61B 5/00; A61B 5/11; A61B 7/00; A61B 7/04; A61F 5/56; G16H 40/67

(52) U.S. Cl. CPC A61B 5/4818; A61B 5/1114; A61B 5/4836; A61B 5/6803; A61B 5/7275; A61B 5/7282; A61B 7/003; A61B 7/04; G16H 40/67; A61B 2562/0219; A61F 5/56

(57) ABSTRACT

A processor of an ear-worn device such as an earbud may receive acceleration data from an accelerometer of the earbud. The processor may determine a position of a body wearing the earbud based on the acceleration data. The processor may determine a pathology of the body. The processor may generate, based on the position of the body and the pathology, a therapy recommendation. The processor may output an indication of the therapy recommendation via the earbud or one or more other devices.

### CROSS-REFERENCE TO RELATED APPLICATIONS

This application claims the benefit of priority from U.S. Provisional Application No. 63/685,322, filed on Aug. 21, 2024, and from U.S. Provisional Application No. 63/685,324, filed on Aug. 21, 2024, and from U.S. Provisional Application No. 63/685,325, filed on Aug. 21, 2024, and from U.S. Provisional Application No. 63/813,222, filed on May 28, 2025, and from U.S. Provisional Application No. 63/813,238, filed on May 28, 2025, and from U.S. Provisional Application No. 63/813,243, filed on May 28, 2025, the entirety of each of which is hereby incorporated by reference.

### BACKGROUND

The sleep position of a person may exacerbate existing pathologies and/or present new health problems. For example, sleeping on the back may negatively impact sleep apneas because the gravitational force associated with lying on the back may cause the jaw, tongue, and soft palate to drop back toward the throat, thereby narrowing the airway and causing breathing restrictions. Therefore, the detection and correction of improper sleep positions may treat pathologies and improve patient health.

### SUMMARY

Systems, methods, devices, non-transitory media, and apparatuses are disclosed for positional sleep therapy using earbuds.

In various embodiments, a system comprising ear-worn devices such as earbuds can provide therapy recommendations based on sleep position. Each earbud may contain an accelerometer that captures acceleration data, which is processed by a processor to determine the sleep position of the body wearing the earbuds. Based on the position and a pathology, the processor generates a therapy recommendation, which is then outputted.

In some embodiments, processing instructions are stored on a non-transitory computer-readable storage medium. When executed by a processor of an ear-worn device such as an earbud, these instructions allow the processor to receive acceleration data from the accelerometer, determine the body's sleep position, identify any pathologies, generate a therapy recommendation, and output the recommendation.

In some embodiments, a method includes receiving acceleration data from an accelerometer of an ear-worn device such as an earbud, determining a body's position based on the acceleration data, determining a pathology of the body, generating a therapy recommendation based on the position and pathology, and outputting the therapy recommendation.

The methods, systems, devices, and apparatuses described may be implemented to improve the functionality of a processor, such as a processor of a specific purpose computer, wearable device, respiratory monitor, and/or a respiratory therapy apparatus. Moreover, the described methods, systems, devices, and apparatus can provide improvements in the technological field of automated detection, management, monitoring, and/or treatment of respiratory conditions, including, for example, sleep disordered breathing.

### BRIEF DESCRIPTION OF THE SEVERAL VIEWS OF THE DRAWINGS

FIG. 1 illustrates a system that uses earbuds to provide positional sleep therapy, in accordance with one embodiment.

FIG. 2 illustrates components of an earbud that provides positional sleep therapy, in accordance with one embodiment.

FIG. 3 illustrates an aspect of the subject matter in accordance with one or more embodiments.

FIG. 4 shows a view of the human ear canal.

FIG. 5 illustrates an aspect of the subject matter in accordance with one embodiment.

FIG. 6 illustrates an aspect of the subject matter in accordance with one embodiment.

FIG. 7 illustrates an aspect of the subject matter in accordance with one embodiment.

FIG. 8 illustrates a logic flow 800 in accordance with one embodiment.

FIG. 9 illustrates a logic flow 900 in accordance with one embodiment.

FIG. 10 illustrates a logic flow 1000 in accordance with one embodiment.

FIG. 11 illustrates a logic flow 1100 in accordance with one embodiment.

FIG. 12 shows a patient tracking device worn by a patient.

FIG. 13 shows a patient tracking device worn by a patient together with a patient interface.

FIG. 14 shows a patient interface in the form of a nasal mask.

FIG. 15A-FIG. 15D illustrate components of a Respiratory Pressure Therapy (RPT) device.

FIG. 16 illustrates a computing system 1600 in accordance with one embodiment.

### DETAILED DESCRIPTION

Embodiments disclosed herein include techniques for using ear-worn devices to provide positional sleep therapy. In some embodiments, one or more wearable devices that include an accelerometer, such as ear-worn devices, are used to detect the sleep position of a person. The sleep position may include a supine position (e.g., sleeping at least partially on the back), a prone position (e.g., sleeping at least partially on the stomach), a lateral sleep position (e.g., sleeping at least partially on the side body), or any combination thereof. Based on the detected sleep position, the wearable devices may generate a therapy recommendation.

For example, an ear-worn device, such as one or more earbuds, may include an accelerometer. A processor of an earbud may use data from the accelerometer to determine that the wearer of the earbuds is sleeping in the supine position. The processor of the earbud may generate a therapy recommendation, such as to change the sleep position, and output an indication to change the sleep position. For example, the earbuds may output sounds or vibrations to cause the person to change their sleep position, e.g., to sleep on their side instead of their back.

In some embodiments, the earbuds may generate the therapy recommendations based on one or more pathologies of the patient. For example, the supine position may negatively impact pathologies such as sleep apnea (e.g., obstructive sleep apnea (OSA), positional sleep apnea (POSA), central sleep apnea (CSA), etc.). Therefore, the earbuds may determine (e.g., based on a user profile), that the person has OSA. Because sleeping on the back may worsen OSA, e.g., cause further breathing restrictions, the earbuds may generate the therapy recommendation based on the determined sleep position and the pathology. Therefore, the earbuds may output sounds, vibrations, etc., to gently cause the person to change their sleep position.

In some embodiments, the sleep position may be multimodal, e.g., may include the positions of different body parts. For example, the sleep position may include one or more of the positions of the head, the torso, the wrist, the arms, the legs, or any combination thereof. In some embodiments, the sleep position of the person is determined by the earbuds based on data received from other devices, such as smartwatches, smart rings, torso-worn devices, implanted devices, and/or devices worn on the legs. Doing so allows the earbuds to more precisely determine the sleep position based on the positions of the different parts of the body. For example, a person may wear a smartwatch on their wrist, where the smartwatch includes an accelerometer. The smartwatch may process data from the accelerometer to determine the orientation of the hand, wrist, and/or arm. The earbuds may receive the orientation information (and/or the raw accelerometer data) from the smartwatch and process the data.

For example, the data from the accelerometers of the earbuds may indicate the head is facing up (e.g., person is sleeping in the supine position). The earbuds may then determine, based on the data received from the smartwatch, that the palm is facing down, which may be used by the earbuds to further determine that the person is sleeping in the supine position. In some embodiments, however, the data received from the smartwatch may indicate the arm, hand, and/or wrist is in an orientation associated with side sleeping. Therefore, the earbuds may determine that the person is sleeping in a combination of positions, e.g., with the head facing at least partially up and the arms and/or torso in a side sleeping position. Based on the precise orientation information determined by the earbuds, the earbuds may generate a therapy recommendation as described herein.

In some embodiments, the data from the accelerometer may be used by the earbuds to determine an angle of inclination of the body relative to the bed as part of the sleep position (e.g., that the person is sleeping at a 30 degree angle relative to their bed). The earbuds may use the determined angle of inclination to provide therapy recommendations. For example, a person may have apneas or hypopneas when sleeping at an angle such as 25 degrees, e.g., when the head of the bed is raised, or the person has several pillows behind their head. Therefore, if the earbuds subsequently determine the person is sleeping at an angle greater than or equal to this angle, the earbuds may generate a therapy recommendation.

Embodiments disclosed may generate any type of therapy recommendation for a detected sleep position. For example, the therapy recommendations may include changing the type of mask used as part of a respiratory therapy system, change operating parameters of the respiratory therapy system (e.g., modifying the therapy pressure provided by Continuous Positive Airway Pressure (CPAP) device), changing the type of respiratory therapy system, educational recommendations such as using fewer (or more) pillows, etc. In some embodiments, the earbuds may programmatically initiate the therapy recommendations, e.g., by transmitting an instruction to a respiratory therapy device to modify one or more operational parameters (e.g., adjusting titration, pressure, etc.), transmitting an indication to a medical provider to prescribe a respiratory therapy device, placing an order for new and/or replacement parts, etc.

In some embodiments, the earbuds may predict events based on the determined sleep position. For example, if a person is sleeping on their back, the earbuds may predict an airway obstruction may occur. The earbuds may therefore generate an audible alert or vibration to cause the patient to stop sleeping on their back.

In some embodiments, the earbuds may detect apneas or hypopneas while a person is sleeping. The earbuds may determine the sleep position of the patient when the apneas or hypopneas are detected. The earbuds may associate each detected apnea or hypopnea with the corresponding sleep position in a user profile. As stated, the sleep position may include the positions of multiple body parts and/or an angle of inclination. Doing so may allow the earbuds to predict events, e.g., apneas or hypopneas, based on the current sleep position and the data in the user profile. For example, if the user profile indicates the person has hypopneas that exceed a threshold while sleeping on their stomach, the earbuds may generate an alert when the person is sleeping on their stomach.

In some embodiments, the earbuds may determine sleep positions based at least in part on detecting sounds. For example, the earbuds may capture soundwaves using one or more microphone arrays, analyze the soundwaves, and determine the sleep position based on the analysis. For example, the soundwaves may reflect that sounds from the mouth (e.g., snoring, coughing, sneezing, etc.) are being muffled by pillows, blankets, etc. Therefore, the earbuds may determine the person is sleeping on their stomach based at least in part on the detected sounds.

In some embodiments, the earbuds may detect the pathologies of the person as part of generating a positional sleep therapy recommendation. For example, the earbuds may include a plurality of microphone arrays to detect sounds and one or more speakers to emit sounds. The earbuds may analyze the sounds and determine a location of the sounds in the patient, e.g., using a position determination algorithm. For example, by detecting snoring sounds, obstructive sleep apnea (OSA) events may be detected. The earbuds may further determine that the sounds are associated with a respiratory therapy device, to determine one or more pathologies being treated by the respiratory therapy device. Therefore, based on detecting pathologies and/or the devices being used by the patient, the earbuds may generate therapy positional sleep therapy recommendations.

Advantageously, embodiments disclosed herein provide techniques to identify, in real time, a sleep position that may negatively impact the health of a person and affect a treatment or prophylaxis thereof. By leveraging sensors integrated into wearable devices that can identify the sleep position of patients, a precise determination of sleep position may be generated (e.g., based on the positions of different parts of the body). When a sleep position is (by itself and/or in combination with a pathology) detrimental to the health of the person, the earbuds may recommend a treatment to improve the health of the patient. For example, embodiments disclosed herein may determine a particular recommendation, e.g., changing sleep positions, modifying parameters for respiratory therapy systems, reordering supplies, and affecting the recommendation to improve the health of the patient. Because some sleep-related pathologies may be severe (or even fatal), the real-time detection of sleep positions may provide opportunities to avoid these outcomes. Embodiments are not limited in these contexts.

Aspects of the present disclosure and certain features, advantages, and details thereof are explained more fully below with reference to the non-limiting examples illustrated in the accompanying drawings. Descriptions of well-known processing techniques, systems, components, etc. are omitted to not unnecessarily obscure the disclosure in detail. The detailed description and the specific examples, while indicating aspects of the disclosure, are given by way of illustration only, and not by way of limitation. Various substitutions, modifications, additions, and/or arrangements, within the spirit and/or scope of the disclosed aspects will be apparent to those skilled in the art from this disclosure. Note further that numerous aspects and features are disclosed herein, and unless inconsistent, each disclosed aspect or feature is combinable with any other disclosed aspect or feature as desired for a particular embodiment of the concepts disclosed herein.

Unless described or implied as exclusive alternatives, features throughout the drawings and descriptions should be taken as cumulative, such that features expressly associated with some particular embodiments can be combined with other embodiments. Like numbers refer to like elements throughout.

While certain exemplary embodiments have been described and shown in the accompanying drawings, it is to be understood that such embodiments are merely illustrative of, and not restrictive on, the broad disclosure, and that this disclosure not be limited to the specific constructions and arrangements shown and described, since various other changes, combinations, omissions, modifications and substitutions, in addition to those set forth in the above paragraphs, are possible. Those skilled in the art will appreciate that various adaptations, modifications, and combinations of the herein described embodiments can be configured without departing from the scope and spirit of the disclosure. Therefore, it is to be understood that, within the scope of the included claims, the disclosure may be practiced other than as specifically described herein.

Additionally, illustrative embodiments are described below using specific code, designs, architectures, protocols, layouts, schematics, or tools only as examples, and not by way of limitation. Furthermore, the illustrative embodiments are described in certain instances using particular software, tools, or data processing environments only as example for clarity of description. The illustrative embodiments can be used in conjunction with other comparable or similarly purposed structures, systems, applications, or architectures. One or more aspects of an illustrative embodiment can be implemented in hardware, software, or a combination thereof.

As understood by one skilled in the art, program code, as referred to in this application, can include both software and hardware. For example, program code in certain embodiments of the present disclosure can include fixed function hardware, while other embodiments can utilize a software-based implementation of the functionality described. Certain embodiments combine both types of program code.

The terms “coupled,” “fixed,” “attached to,” “communicatively coupled to,” “operatively coupled to,” and the like refer to both (i) direct connecting, coupling, fixing, attaching, communicatively coupling; and (ii) indirect connecting coupling, fixing, attaching, communicatively coupling via one or more intermediate components or features, unless otherwise specified herein. “Communicatively coupled to” and “operatively coupled to” can refer to physically and/or electrically related components.

Some of the figures may include a logic flow. Although such figures presented herein may include a particular logic flow, it can be appreciated that the logic flow merely provides an example of how the general functionality as described herein can be implemented. Further, a given logic flow does not necessarily have to be executed in the order presented unless otherwise indicated. Moreover, not all acts illustrated in a logic flow may be required in some embodiments. In addition, the given logic flow may be implemented by a hardware element, a software element executed by a processor, or any combination thereof. The embodiments are not limited in this context.

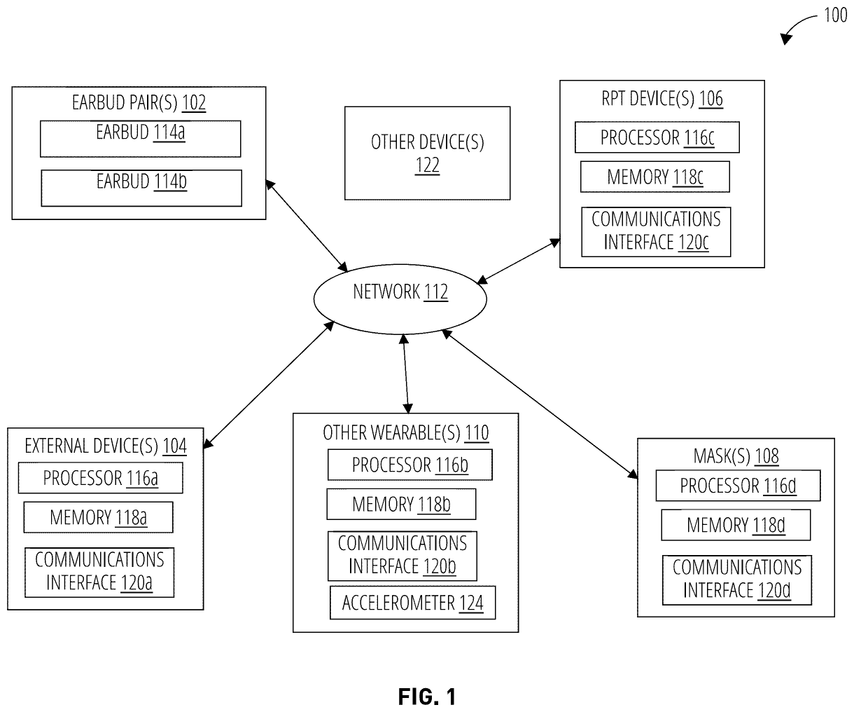

FIG. 1 illustrates a system 100 in accordance with one embodiment. The system 100 may be a system that uses earbuds to provide positional sleep therapy. Therefore, one or more components of the system 100 may be part of a patient health system. Embodiments are not limited in these contexts.

As shown, the system 100 includes one or more earbud pairs 102, one or more external devices 104, one or more respiratory therapy (RPT) devices 106, one or more masks 108, and one or more other wearables 110 communicably coupled via a communications network 112. In some embodiments, the system 100 includes one or more other therapy devices 122, e.g., intraoral therapy devices, mouth tape, teeth guards, nerve stimulation devices (e.g., transcutaneous electrical nerve stimulation (TENS) devices, percutaneous electrical nerve stimulation devices etc.), or any other therapy device.

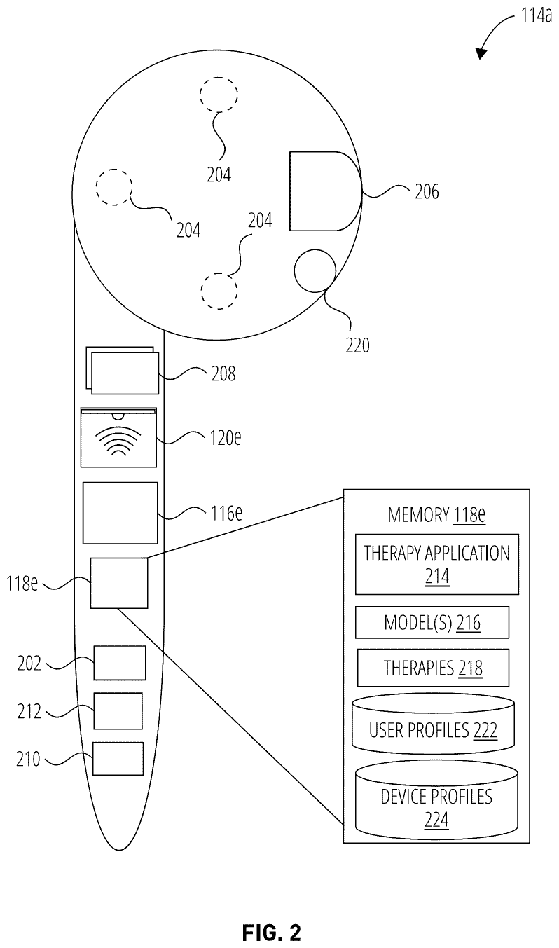

The earbud pair 102 includes an earbud 114a and an earbud 114b. Additional components of the earbuds 114a-114b are depicted in FIG. 2. Generally, earbuds 114a-114b are worn in, around, or proximate to the ear of a person. Although the “earbud” is used as one reference example herein, the disclosure is equally applicable to other types of ear-worn electronic devices. Therefore, embodiments are not limited to the earbud form factor.

The external devices 104 represent any type of device, such as a computing device, smartphone, laptop, tablet, hub, smart home device, medical provider device or system, medical device, networking device, Internet of things (IoT) device, and the like. The other wearables 110 represent any type of wearable device, such as smartwatches, devices worn on the torso (e.g., heartrate monitors), smart rings, smart goggles, smart glasses, step counters, medical devices, straps, ankle or leg-worn devices, and the like.

The RPT device 106 represents any respiratory therapy device or system, such as a Continuous Positive Airway Pressure (CPAP) device. More generally, the RPT device 106 is configured to generate a flow of air for delivery to the human airways via an interface such as a mask 108. In some embodiments, the RPT devices 106 include RPT devices that are implanted at least partially within the body of a patient.

As shown in FIG. 1, the external devices 104, RPT devices 106, masks 108, and other wearables 110 include a processor 116a, a processor 116b, a processor 116c, and a processor 116d, respectively. As shown in FIG. 2, the earbud 114a, which represents earbud 114b, similarly includes a processor 116e, a memory 118e, and a communications interface 120c. Furthermore, the other wearables 110 include an accelerometer 124, while the earbuds 114a-114b each include a respective accelerometer 212. An accelerometer such as accelerometer 124 or accelerometer 212 may measure the rate of change of velocity (e.g., acceleration) of the device (e.g., the other wearables 110 and earbuds 114a, 114b, respectively) along one or more axes, providing data on movement and orientation.

The processors 116a-116e represent any type of processor circuit. Examples of processor circuits include an Intel® x86 processor, an ARM® processor, a 32-bit RISC CPU, a 16-bit RISC CPU, AMD® processors, and similar processors. Similarly, the external devices 104, RPT devices 106, masks 108, and other wearables 110 include a memory 118a, a memory 118b, a memory 118c, and a memory 118d, respectively. The memories 118a-118e represent any type of computer memory, such as volatile memory or non-volatile memory. To communicate via the network 112, the external devices 104, RPT devices 106, masks 108, and other wearables 110 include a communications interface 120a, a communications interface 120b, a communications interface 120c, and a communications interface 120d, respectively. The communications interfaces 120a-120e represent any type of data communications interface, such as a wireless (or wired) transceiver. In some embodiments, the other therapy devices 122 similarly include a processor, memory, and communications interface to the network 112.

The network 112 may be any type of data communications network. In some embodiments, the network 112 is a wireless communications network. Examples of wireless communications networks include an IEEE 802.11 wireless network, Wi-Fi, Bluetooth®, Bluetooth Low Energy (BLE), near-field communication (NFC), radio frequency identification (RFID), radio frequency (RF) networks, or any other type of wireless communication network. Therefore, the communications interfaces 120a-120e are configured to support IEEE 802.11 wireless networks, Wi-Fi, Bluetooth, Bluetooth Low Energy (BLE), near-field communication (NFC), radio frequency identification (RFID), radio frequency (RF) networks, or any other type of wireless communication network. Furthermore, the network 112 represents direct wireless communications between the entities of the system 100.

Collectively, the components of the system 100 (or any subset thereof) are configured to monitor a human patient, collect data from the patient, determine a sleep position of the patient, deliver therapy (e.g., a treatment) to the patient, detect therapies delivered to the patient, detect respiratory therapy systems (and/or components thereof) used to deliver therapies to the patient, modify therapies delivered to the patient, and/or generate recommendations.

For example, the earbuds 114a-114b may collect data from the patient, determine a sleep position of the patient, determine a pathology of the patient that may be exacerbated by the sleep position, and generate one or more therapy recommendations. In some embodiments, the collected data may include data from the external devices 104, other wearables 110, masks 108, and/or RPT devices 106. In some embodiments, the therapy recommendation is outputted by the earbuds 114a-114b to the patient, e.g., as an audible alert, vibrations, etc. In some embodiments, the therapy recommendation is outputted by other devices of the patient, e.g., smartphones, smartwatches, etc. Stated differently, any recommendation or other content disclosed herein may be outputted via the external devices 104, RPT devices 106, other wearables 110 and/or masks 108.

In some embodiments, the therapy recommendation is transmitted by earbud 114a or 114b via the network 112. For example, the therapy recommendation may include prescribing a different type of mask 108, prescribing a different type of RPT device 106, modifying parameters of the RPT device 106 (e.g., changing pressure, titration, etc.), changing bed types, changing the number or type of pillows used while sleeping, etc. In some embodiments, the therapy recommendation may be sent to an external device 104, such as a medical provider system. Doing so allows the medical provider to prescribe a therapy for the patient, e.g., to prescribe a different type of mask 108, change RPT devices 106 (e.g., CPAP, bilevel, etc.), change attributes of the therapy provided by the RPT device 106, etc. In another example, the alert may be sent as an instruction to the RPT device 106 and/or the mask 108. For example, the RPT device 106 may modify, based on the instruction, the type of therapy, attributes of the therapy (e.g., increase pressure, decrease pressure, modify titration, etc.), and/or a duration of the therapy provided to the patient. Embodiments are not limited in these contexts.

FIG. 2 illustrates an example earbud 114a that can communicatively couple with earbud 114b to form a pair of untethered, wireless earbuds according to some embodiments of the present technology. Although earbud 114a is depicted, earbud 114b includes the components depicted in FIG. 2.

The earbud 114a uses communications interface 120e to communicatively couple with another wireless earbud, e.g., earbud 114b, and to pair with a source device, e.g., a companion communication device (e.g., external devices 104 such as smartphones) that can provide audio data that the earbuds 114a can reproduce as audio signals for a user of the earbuds 114a, 114b. In some embodiments, a process of pairing the earbuds 114a, 114b is initiated when the earbuds 114a, 114b are contained within a housing/case, not pictured for clarity. In some circumstances, once a pairing mode is enabled for the earbuds 114a, 114b, the earbuds 114a, 114b remain in the enabled pairing mode until one or more of the following occurs: (i) the earbud 114a or 114b pairs with a companion communication device, (ii) a pairing mode of the earbuds 114a, 114b times out (e.g., the earbud 114a or 114b does not pair with a companion communication device within a fixed time period, such as thirty seconds), (iii) the earbud 114a or 114b is removed from the case, (iv) the wireless earbud case commands one or more both of the earbuds 114a, 114b to exit the pairing mode, or (v) the companion communication device commands the earbuds 114a, 114b to exit the pairing mode. The earbud 114a can also include a battery 202 and sensors 204 for detecting a wearing status of the earbud 114a, e.g., when the earbud 114a is placed in and/or removed from an ear, whether the earbud 114a is in a user's ear, e.g., an in-ear wearing status, or is not in a user's ear, e.g., an out-of-ear wearing status.

Additionally, the earbud 114a includes an audio output device such as a speaker 206 for converting a received signal, e.g., which can include audio data, into audible sound. The signal can be received from a paired companion communication device via the communications interface 120c. The memory 118e in the earbud 114a stores firmware for operating the earbud 114a as well as data for coupling with other wireless earbuds and for pairing the earbud 114a with companion communication devices. For example, the memory 118e in the earbud 114a can store a connection history for companion communication devices with which the earbud 114a has previously paired. The connection history can include data for automatically pairing the earbud 114a with the companion communication device without having to configure a connection between the earbud 114a and the companion communication device (e.g., enter a password, exchange shared secrets, etc.). For example, the connection history can include one or more link keys for connecting to a wireless network such as network 112 (e.g., Bluetooth link keys). The memory 118e of the earbud 114a can also store a MAC address that uniquely identifies the earbud 114a as well as store a paired partner MAC address of another wireless earbud 114b that has previously coupled with the earbud 114a. The memory 118e also stores instructions that, when executed by the processor, causes the earbud 114a to communicatively couple with another wireless earbud.

As shown, the earbud 114a includes one or more sensors 204, one or more speakers 206, two or more microphone arrays 208, a haptic feedback module 210, an accelerometer 212, and a pulse oximeter 220. The speakers 206 are devices to output audio, e.g., soundwaves. Each of the microphone arrays 208 includes a plurality of microphones (not pictured) that are configured to detect and record audio data, e.g., soundwaves. Therefore, a given earbud 114a, 114b, may include a plurality of microphone arrays 208, with each microphone array 208 including a plurality of microphones. The total number of microphones in each earbud 114a, earbud 114b may, therefore, number in the tens, hundreds, thousands, or more. In some embodiments, a first one of the microphone arrays 208 is located at a first end of the earbud 114a (e.g., nearest to the ear canal), while a second one of the microphone arrays 208 is located at an opposite end of the earbud 114a (e.g., farthest from the ear canal). In such embodiments, one or more other microphone arrays 208 may be located between the first and second microphone arrays 208. Embodiments are not limited in these contexts.

The haptic feedback module 210 is a device that generates vibrations or other tactile sensations, such as piezoelectric actuators/sensors, etc. The haptic feedback module 210 may detect reflections thereof, e.g., reflections of vibrations from the ear canal when the earbud 114a is worn by a patient, which may be useful in detecting a pathology in the patient or detecting a therapy device used by the patient. The haptic feedback module 210 may further output vibrations or other haptic feedback to cause a patient to change sleep positions.

The sensors 204 represent any type of sensor, such as a pressure sensor, a flow rate sensor, a temperature sensor, a motion sensor, a camera, an infrared (IR) sensor, a photoplethysmogram (PPG) sensor, an electrocardiogram (ECG) sensor, an electroencephalography (EEG) sensor (e.g., an electrode), a capacitive sensor, an electromyography (EMG) sensor, an oxygen sensor, an analyte sensor, a moisture sensor, a light detection and ranging (LiDAR) sensor, an electrooculography (EOG) sensor, a galvanic skin response (GSR) sensor, or a carbon dioxide (CO2) sensor. The pulse oximeter 220 is a peripheral oxygen saturation sensor that is configured to determine a peripheral oxygen saturation (SpO₂) value of a bloodstream of a patient. Stated differently, the pulse oximeter 220 is configured to detect the oxygen levels of a patient.

As shown, the memory 118e of the earbud 114a includes a therapy application 214, one or more models 216, a data store of therapies 218, a data store of user profiles 222, and a data store of device profiles 224. The therapy application 214 is generally configured to determine the sleep position of a person based at least in part on the data from the accelerometer 212 of the earbud 114a and/or the accelerometer 212 of earbud 114b. The therapy application 214 may further determine therapy recommendations based on the sleep position of the person and/or one or more pathologies of the person. The pathologies of the person may be specified in the user profile 222 and/or programmatically detected as described herein. More generally, the therapy application 214 may detect pathologies in a patient, detect therapy devices worn or otherwise used by a patient, generate models of the ear canal of the patient, predict pathologies of the patient, determine the position of sounds, track patient adherence to therapy, detect errors or configuration issues with therapy devices, and/or generate recommendations.

The user profiles 222 store a plurality of attributes for one or more users. For example, the user profiles 222 may store indications of sleep positions detected by the therapy application 214 (with corresponding timestamps), data recorded by the sensors 204 (e.g., oxygen saturation values, respiratory rate, etc.), pathologies associated with the user, devices used by or otherwise prescribed to the user (e.g., RPT device 106, a mask 108, etc.), models of the ear canal of the user, use of the devices prescribed to the user (e.g., a log of entries detailing dates and times when the user uses their mask 108, RPT device 106, etc.), detected apneas (which may be associated with a timestamp and a detected sleep position when the apneas occur), detected hypopneas (which may be associated with a timestamp and a detected sleep position when the hypopneas occur), or any other attribute of the user. The device profiles 224 include data describing different devices, such as RPT devices 106, masks 108, other wearables 110, other therapy devices 122, external devices 104, etc. Example attributes stored in the device profiles 224 include device type, device model, device function, sound profiles, how the device is worn or otherwise used by the patient, configurations, associated components, and the like. The device profiles 224 may further include associations between devices (e.g., RPT devices 106, masks 108, other wearables 110, other therapy devices 122, etc.) and one or more pathologies for which the devices are prescribed to provide therapy. The device profiles 224 may further include indications of sleep positions that negatively impact a pathology and/or the ability of a device to deliver therapy.

As stated, the accelerometer 212 (which is representative of the accelerometers 124 of the other wearables 110) is a device that measures the rate of change of velocity (e.g., acceleration) of the earbud 114a along three orthogonal axes (X, Y, and Z, in three-dimensional space), providing data on movement and orientation of the earbud 114a. The data provided by the accelerometer 212 may therefore be acceleration data in units of meters per second squared (m/s²) (or “g”, where 1 g is approximately 9.8 m/s², the acceleration due to the Earth's gravity). In some embodiments, the accelerometer 212 provides data reflecting static forces (e.g., the pull of gravity when the earbud 114a is stationary, which may be used to determine tilt and/or orientation) and dynamic forces (e.g., forces from motion or vibration, which may be used to detect movement patterns).

The therapy application 214 executing on processor 116e of earbud 114a may use the acceleration data from the accelerometer 212 to determine position, e.g., by computing a first integration of the acceleration data over time to determine velocity. The therapy application 214 may compute a second integration of the velocity to determine the position of the earbud 114a. Therefore, using the data from the accelerometer 212, the therapy application 214 detects acceleration, velocity, and/or movement.

To determine the sleep position of a person wearing the earbuds 114a, 114b, the therapy application 214 may process the sensor data along each of the X, Y, and Z axes from the accelerometer 212 over one or more time intervals (e.g., milliseconds, seconds, minutes, etc.). In some embodiments, a baseline calibration is performed using the accelerometer 124, e.g., to determine the orientation when the person is standing upright, sitting, etc., to calibrate the X, Y, and Z axes relative to gravity.

For example, if the sensor axis pointing upward shows a dominant gravitational pull (e.g., the Z-axis is approximately 9.8 m/s², and the X and Y axes are approximately zero), the therapy application 214 may determine the person is sleeping on their back (e.g., in a supine position). As another example, if the sensor axis pointing downward shows a dominant gravitational pull (e.g., the Z-axis is approximately −9.8 m/s², and the X and Y axes are approximately zero), the therapy application 214 may determine the person is sleeping on their stomach (e.g., in a prone position). Further still, if the data from the accelerometer 124 reflects minimal acceleration on the Z-axis but a strong signal on the X-axis (or Y-axis, depending on the assignment of axes), the therapy application 214 may determine the person is sleeping on their side. The particular side that the person is sleeping on may be based on the detected forces. For example, if the X-axis is approximately 9.8 m/s², and the Y and Z axes are approximately zero, the therapy application 214 determine the person is lying on their right side. As another example, if the X-axis is approximately −9.8 m/s², and the Y and Z axes are approximately zero, the therapy application 214 may determine the person is lying on their left side.

Similarly, the processors 116b of the other wearables 110 may compute the first and second integrations based on the acceleration data from the accelerometers 124 to determine the respective positions of the other wearables 110. Further still, the processors 116b of the other wearables 110 may perform the same X, Y, and Z axis processing to determine whether the corresponding body part is facing up, down, or to the side (and to which side based on the particular configuration). However, in some embodiments, the raw sensor data from the accelerometers 124 of the other wearables 110 may be transmitted to the therapy application 214, which may determine the position of the other wearables 110, and the associated body part, as described above.

Since the earbuds 114a, 114b are worn in the ears of the person, in some embodiments, the sleep position determined by the therapy application 214 based on the data from the accelerometers 212 may be associated with the position of the person's head. In some embodiments, the position of the head may be considered to be the sleep position of the person.

As stated, in some embodiments, the sleep position may be based on data from the other wearables 110. For example, in some embodiments, the therapy application 214 may receive the position data from the other wearables 110 and base the determination of the sleep position based on the received data.

For example, if the other wearables 110 include a chest strap monitor worn on the person's chest, the processor 116b may use data from the accelerometer 124 to determine the orientation of the chest strap monitor. Because the chest strap monitor is worn on the chest, the data from the accelerometer 124 therefore indicates the orientation of the chest. Therefore, the chest strap monitor may provide the orientation data (and/or the raw sensor data from the accelerometer 124) to the therapy application 214 of the earbud 114a via the network 112. The therapy application 214 may therefore further determine the sleep position of the person based on the data from the chest strap monitor (and chest or torso by association).

For example, if the therapy application 214 determines the person is sleeping in a supine position, while the chest strap monitor indicates the chest is pointing upward (e.g., the accelerometer 124 data from the chest strap monitor indicates the Z-axis is approximately 9.8 m/s², and the X and Y axes are approximately zero), the therapy application 214 may determine (to a greater degree of confidence) that the person is sleeping on their back.

However, as stated, in some embodiments, the sleep position is multi-modal, e.g., reflects the position of different body parts. Therefore, the therapy application 214 may collect data from the other wearables 110 to determine the orientation of the associated part of the body the other wearables 110 are worn on or otherwise proximate to. Continuing with the previous example, the therapy application 214 may determine the person's head is facing upward (e.g., in the supine position) and the torso is facing upward (e.g., in the supine position).

Similar determinations may be made for other wearables 110, e.g., for smartwatches worn by the person, devices worn on the legs (e.g., ankle step trackers, etc.), devices worn on the hips, etc. Generally, the accelerometer 124 of any of the other wearables 110 may provide data used to determine an orientation and/or position of the device (and body part, by association) as described herein. For example, if the data from the earbuds 114a-114b, smartwatch, leg-worn devices indicate the person is sleeping in a combination of sleep positions (e.g., head in a first orientation, torso in a second orientation, legs, in a third orientation, etc.), which may result in poor posture of the spine, the therapy application 214 may generate an alert to cause the person to change sleep position.

Similarly, as stated, some RPT devices 106 and/or other therapy devices 122 may be implanted at least partially within the body. These devices may include accelerometers that provide data to further enhance the precision of a sleep position determination and/or to determine the position of the body part where the device is implanted as part of the sleep position determination. In some embodiments, these implanted devices operate most effectively in one or more particular sleep positions. Therefore, the therapy application 214 may determine when a person with an implanted device is not sleeping these particular sleep positions and generate an alert to cause the person to change to one of these sleep positions.

As another example, the therapy application 214 may determine, based on the data from the accelerometer 212, that the forces are in the X or Y axes, with minimal or no forces in the Z position, indicating the face is turned to the side (e.g., one side of the face against the pillow or bed). Furthermore, the chest strap monitor may indicate the torso is facing down (e.g., forces are negative on the Z axis, with minimal or no forces on the X or Y axes). Therefore, based on these determinations, the therapy application 214 may determine the person is sleeping on their stomach.

More generally, using the disclosed techniques, the therapy application 214 may continuously monitor the sleep position of the patient, e.g., at predetermined time intervals. Doing so may advantageously detect sleep positions that may negatively impact the person's health. In response, the therapy application 214 may generate one or more therapy recommendations. In some embodiments, the therapy application 214 references the therapies 218, which includes associations between one or more sleep positions and one or more therapies or treatments. For example, the therapies 218 may indicate, for a back sleeper, to output sounds via the speakers 206 and/or vibrations via the haptic feedback modules 210 to wake the person. Doing so may cause the person to change sleep positions, e.g., from the back to the side. In some embodiments, the therapy recommendations may include audible instructions outputted by the speakers 206, e.g., spoken words instructing the person to change sleep position. In some embodiments, the therapy application 214 may output the recommendations at periodic intervals (e.g., every 30 seconds, 1 minute, etc.) until the therapy application 214 determines the user has changed sleep positions.

In some embodiments, the therapy application 214 determines one or more pathologies of the patient, e.g., based on the user profiles 222, the microphone arrays 208 detecting sounds associated with therapy devices such as RPT device 106 and/or masks 108, etc. The therapy application 214 may therefore analyze the sleep position and/or the identified pathologies to determine the person's health may be negatively affected by the sleep position. For example, if the person has positional sleep apnea associated with a particular sleep position, and the therapy application 214 determines that the person is sleeping in that particular position, the therapy application 214 may generate one or more therapy recommendations. For example, the therapies 218 may indicate to output sounds via the speakers 206 and/or vibrations via the haptic feedback modules 210 to wake the person. Doing so may cause the person to change sleep positions.

As another example, if the person has OSA, sleeping on the back may exacerbate apneas and/or hypopneas. Therefore, based on a determination that the person is sleeping at least partially on their back (e.g., the head and/or torso are facing upward) and has OSA, the therapy application 214 may determine to generate a therapy recommendation. Therefore, in some embodiments, the therapies 218 includes associations between one or more sleep positions, one or more pathologies, and one or more therapies or treatments. For example, the therapies 218 may specify, for the OSA patient sleeping on their back, to modify the therapy provided by the RPT device 106. The modification of the therapy may include modifying pressure, changing pressure mode (e.g., fixed pressure mode and/or auto-adjusting (APAP) pressure mode), ramp time, pressure titration, humidification, tidal volume, respiratory rate, inspiratory time, rise time, etc. In some embodiments, the therapy application 214 may transmit an instruction to the RPT device 106 to implement the therapy modifications identified in the therapies 218 in real-time. In some embodiments, the therapy application 214 may transmit an indication of the modifications to the patient's medical provider, e.g., to change the person's prescription and modify the RPT device 106 accordingly.

More generally, the therapy application 214 may generate any number and type of therapy recommendations. For example, the therapy recommendations may include changing the type of mask 108 used by the patient, providing educational recommendations such as using fewer (or more) pillows, changing mattress type (e.g., a firmer mattress, a softer mattress, etc.), displaying one or more pages of the instruction manual for the RPT device 106 and/or mask 108 on the user's smartphone to assist the user to properly wear the devices, etc.

The therapy application 214 may output notifications, recommendations, or any other types of content via the earbuds 114a-114b, the external devices 104 (e.g., the user's smartphone), the RPT devices 106 of the user, the other wearables 110 of the user (e.g., the user's smartwatch), and/or masks 108 of the user. For example, the therapy application 214 of earbuds 114a-114b may transmit an instruction or other indication of the content to be outputted to the external devices 104 of the user, external devices 104, RPT devices 106, masks 108, and/or other wearables 110, e.g., via the network 112.

In some embodiments, the data from the accelerometers 212 may be used by the therapy application 214 to determine an angle of inclination of the body relative to the bed as part of the sleep position (e.g., that the person is sleeping at a x-degree angle relative to their bed). In some embodiments, accelerometer 212 calibration may occur when the person is lying flat on the bed, which provides the therapy application 214 a reference for the neutral position. The gravitational force vector components (aₓ, a_{y}, a_{z}) from the accelerometer 212 may be used to compute the tilt angles (e.g., pitch for front to back tilt, roll for side to side tilt) of the earbud 114a, 114b. For example, pitch angle θ may be computed according to the following equation:

$$θ = arc tan ( \frac{{a}_{x}}{\sqrt{{a}_{y}^{2}} + {a}_{z}^{2}} ) .$$

Similarly, roll ϕ may be computed according to the following equation:

$$ϕ = arctan ( \frac{{a}_{y}}{\sqrt{{a}_{x}^{2} + {a}_{z}^{2}}} ) .$$

Therefore, the therapy application 214 may use the determined angles to provide therapy recommendations. For example, a person may have apneas or hypopneas when sleeping at a pitch angle such as 25 degrees, e.g., when the head of the bed is raised, or the person has several pillows behind their head. Therefore, if the therapy application 214 subsequently determines the person is sleeping at a pitch angle greater than or equal to 25 degrees, the therapy application 214 may generate a therapy recommendation. For example, the therapy recommendation may indicate to lower the bed, remove one or more of the pillows, etc., to reduce the angle below 25 degrees.

In some embodiments, the therapy application 214 may predict events based on the determined sleep position. For example, if a person is sleeping on their back, the therapy application 214 may predict that an airway obstruction may occur. The therapy application 214 may therefore generate an audible alert or vibration to cause the patient to stop sleeping on their back.

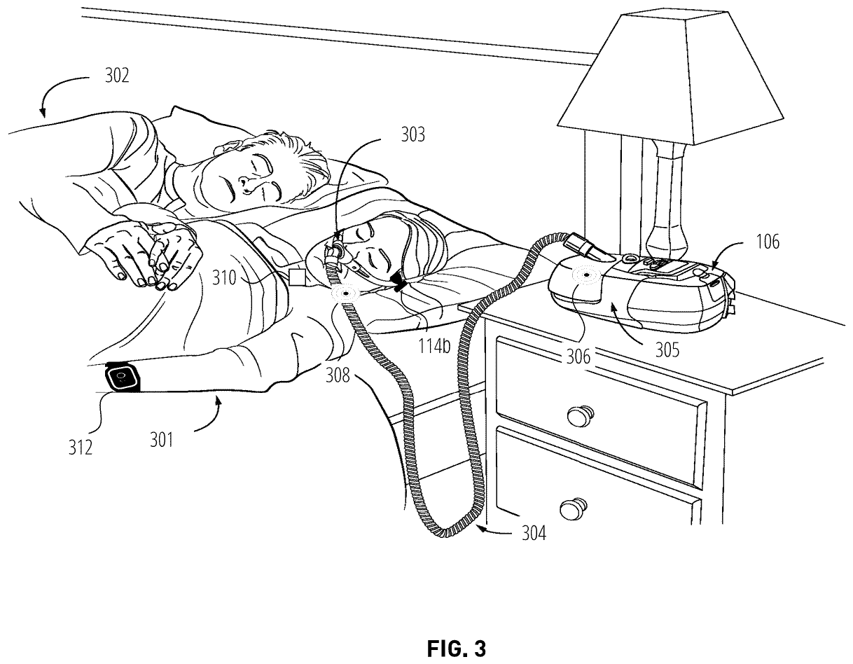

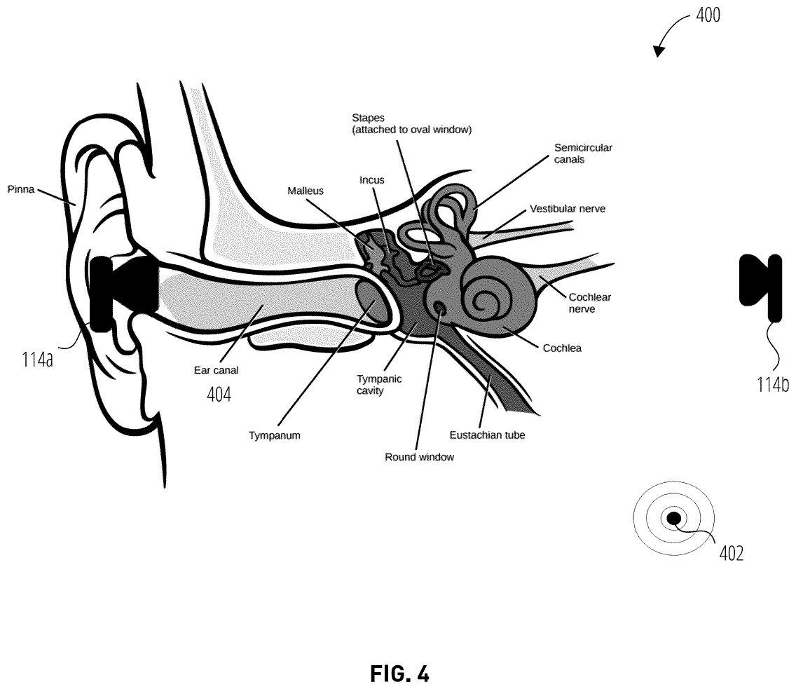

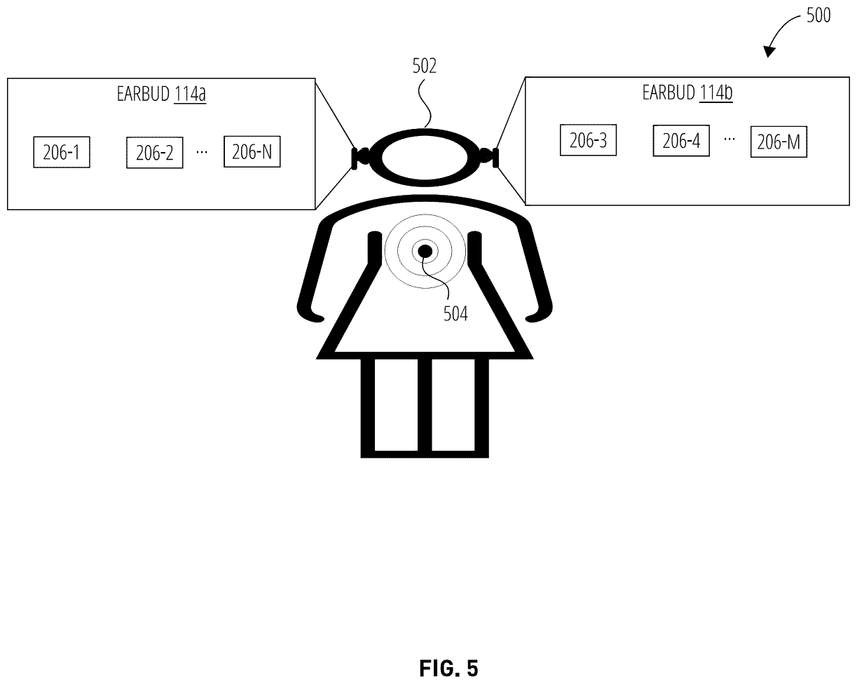

In some embodiments, the therapy application 214 uses sound analysis for positional sleep therapy. Generally, the microphone arrays 208 of an earbud 114a, 114b may capture sounds and determine the type and location of the sound. Examples of such sounds include soundwaves 306, 308, 402, and 504, of FIG. 3, FIG. 4, and FIG. 5, respectively. For example, the therapy application 214 may detect snoring sounds, detect sounds associated with airway collapses in an airway of the person, detect therapy devices such as RPT device 106 and/or mask 108, etc.

Therefore, in some embodiments, the therapy application 214 may detect apneas or hypopneas while a person is sleeping, e.g., based on detecting sounds associated with apneas and/or hypopneas. The therapy application 214 may determine the sleep position of the patient when the apneas or hypopneas are detected. The therapy application 214 may associate each detected apnea or hypopnea with the corresponding sleep position in an entry of the user profile 222 of the person. As stated, the sleep position may include the positions of multiple body parts and/or an angle of inclination. Doing so allows the therapy application 214 to predict events, e.g., apneas or hypopneas, based on the current sleep position and the data in the user profile 222. For example, if the user profile 222 indicates the person has hypopneas that exceed a threshold while sleeping on their stomach, the therapy application 214 may generate an alert when determining the person is sleeping on their stomach.

As stated, in some embodiments, the therapy application 214 may store indications of detected pathologies in the user profiles 222. For example, when apnea events are detected, the therapy application 214 may store indications of each apnea event, the type of apnea event (e.g., OSA, positional sleep apnea, central sleep apnea, etc.), a timestamp, and a sleep position of the patient. In some embodiments, the therapy application 214 may compute scores, ratios, or other metrics associated with the data in the user profiles 222. For example, the therapy application 214 may compute a score for the patient based on a ratio of sleep apneas that are associated with a specific sleep position (e.g., positional sleep apnea detected while the patient is sleeping on their back) and apneas that are not associated with a sleep position (e.g., apneas detected when the patient is sleeping in a non-supine position). In some embodiments, the score may reflect the type of sleep apnea. For example, if the ratio of positional sleep apneas (e.g., apneas detected while the patient is sleeping in a supine position) to non-positional sleep apneas (e.g., apneas detected while the patient is sleeping in a non-supine position) exceeds a predetermined threshold, the therapy application 214 may determine the patient has positional sleep apnea. If, however, the ratio is below the threshold, the therapy application 214 may determine the patient has obstructive sleep apnea. Embodiments are not limited in these contexts.

As another example, the therapy application 214 may analyze sounds captured by the microphone arrays 208 and determine the sounds are associated with a partial airway obstruction (e.g., a hypopnea). The therapy application 214 may determine the sleep position of the person when the hypopnea is detected (e.g., the person is sleeping on their back). Based on the detection of the hypopnea and the sleep position, the therapy application 214 may generate a therapy recommendation. For example, the therapy application 214 may output a notification, alert, sound, vibration, etc., to cause the person to change sleep positions.

In some embodiments, the therapy application 214 may determine that the sleep position of the person is interfering with the delivery of respiratory therapy by the RPT device 106 and/or mask 108. For example, by analyzing soundwaves detected by the microphone arrays 208, the therapy application 214 may detect an airflow obstruction that is at least partially obstructing the flow of air to the person. For example, a mask 108 may have one or more conduits, or tubes, which deliver pressurized air therapy to the person. The sleep position of a patient may cause these tubes to be obstructed, e.g., when the person's sleep position impinges or otherwise restricts the flow of air through the tubes. As such, the therapy application 214 may output an indication to cause the patient to change sleep positions, e.g., by outputting sounds and/or vibrations.

In some embodiments, the therapy application 214 may use soundwaves detected by the microphone arrays 208 to generate a model of the ear canal to detect pathologies. In some embodiments, at least a portion of the detected soundwaves are reflections of vibrations or other sounds emitted into the ear canal by the speakers 206 and/or haptic feedback modules 210.

In some embodiments, the therapy application 214 uses additional information collected by the sensors 204 to determine a sleep position. For example, the pulse oximeter 220 may record oxygen saturation (SpO₂) values from the bloodstream of the patient at predetermined intervals (and/or based on instructions from the therapy application 214, e.g., when the therapy application 214 detects a sleep position, a sound, detects a therapy device, detects a pathology, etc.). In some embodiments, the oxygen saturation values are stored in the user profile 222 of the patient. The therapy application 214 may use the oxygen saturation values to determine, at least in part, a sleep position of the patient. For example, if the patient's oxygen saturation is below a threshold, the therapy application 214 may determine that the patient is sleeping on their back (e.g., because the patient is not experiencing suitable SpO₂ levels). The thresholds may be any type of threshold, such as predetermined minimum/maximum oxygen saturation thresholds, thresholds that are associated with a specific patient (e.g., patient's average oxygen saturation while sleeping on their back, side, or stomach, etc.), thresholds associated with a group of patients (e.g., patients with a specific type of sleep position, pathology, etc.). In some embodiments, the thresholds are stored in a patient's user profile 222.

The therapy application 214 and/or models 216 may generally use any location (or position) determination algorithm to determine the location where a sound detected by the microphone arrays 208 originated. Because multiple microphone arrays 208 are included in an earbud pair 102 (whether in a single earbud 114a, 114b or across both earbuds 114a, 114b), positions may be determined using measurements from these fixed points to compute the precise location a sound originated, e.g., using triangulation, trilateration, beamforming, single or multiple microphone acoustic impedance measurements, impulse/frequency response function measurements, etc.

For example, when the plurality of microphone arrays 208 detect a sound, the therapy application 214 and/or the models 216 may receive indications of the sounds (e.g., waveforms) from the microphone arrays 208. The therapy application 214 and/or the models 216 may determine a position of the sound source by measuring the time differences of arrival (TDOA) of the sounds experienced by the microphone arrays 208. As another example, the therapy application 214 and/or the models 216 may perform a mathematical cross-correlation operation that measures the similarity between two detected signals as a function of the time-lag applied to one of them. By shifting one signal in time and calculating the correlation at each shift, the cross-correlation allows the therapy application 214 and/or the models 216 to identify the time offset that maximizes the similarity between the two signals. Although discussed with reference to the therapy application 214 and/or the models 216, the microphone arrays 208 may include logic to perform the position and/or location determination described herein. Similarly, the external devices 104 may include instances of the therapy application 214, models 216, therapies 218, and user profiles 222, e.g., to perform the processing described herein (e.g., based on receiving data collected by the earbuds 114a, 114b).

Furthermore, the microphone arrays 208, the processor 116e, the therapy application 214, and/or the models 216 may analyze the sounds to identify one or more attributes of the detected sounds (e.g., pressure, amplitude, wavelength, and/or frequency). Doing so may be useful to identify the sounds (and/or causes thereof), e.g., sounds associated with therapy devices, sounds associated with pathologies, airway obstructions, etc., as described herein. For example, the pressure, amplitude, wavelength, and/or frequency may be compared to one or more known sounds, e.g., to identify a known sound that is similar to the detected sound. As stated, the known sounds may be stored or otherwise reflected in the models 216 and/or the device profiles 224. Similarly, the models 216 may consider the attributes of the sound and return a known sound as being similar to the detected sounds. The known sounds may be stored by the therapy application 214, e.g., in the device profiles 224. Similarly, the known sounds and/or attributes thereof (e.g., pressure, amplitude, wavelength, frequency, etc.) may be stored as features in the models 216. Doing so allows the therapy application 214 and/or models 216 to match a detected sound to a known sound (e.g., based on one or more of pressure, amplitude, wavelength, frequency, etc.). For example, the therapy application 214 may analyze a sound and determine the sound is associated with an airway obstruction. Doing so allows the therapy application 214 to generate a positional sleep therapy recommendation, e.g., based on the detected airway obstruction and the sleep position of the person.

In some embodiments, a device may be associated as a treatment for one or more pathologies in the device profiles 224. As such, the therapy application 214 and/or models 216 may identify a pathology associated with a detected device and/or sound. For example, the therapy application 214 and/or models 216 may receive a detected sound as input (including any attributes thereof). The therapy application 214 and/or models 216 may identify a known sound, such as the sounds made by an RPT device 106 that are stored in the device profiles 224 and/or models 216, and determine a patient is using the RPT device 106. The therapy application 214 and/or models 216 may identify a corresponding pathology based on the detected device, e.g., RPT device 106. The therapy application 214 and/or models 216 may further identify a therapy 218 associated with the detected device and/or pathology, such as changing one or more parameters of the RPT device 106 providing therapy to the patient.

In some embodiments, distances between respective pairs of the microphone arrays 208 in a given earbud 114a are stored in the memory 118c (e.g., in the therapy application 214, the models 216, etc.) to facilitate the detection of pathologies in a patient, e.g., for use in a location detection algorithm such as triangulation, beamforming, trilateration, single or multiple microphone acoustic impedance measurements, impulse/frequency response function measurements, etc. Similarly, the distance between earbud 114a and earbud 114b may be determined at predetermined time intervals such that both earbuds 114a, 114b, can be used to detect sounds (e.g., associated with devices worn by the patient and/or pathologies of the patient) over time. For example, the processor 116e of earbud 114a may cause the communications interface 120c of the earbud 114a to emit one or more radio signals to earbud 114b. The processor of earbud 114b may determine the distance to earbud 114a based on the radio signals (and any data included in the signals), e.g., based on one or more of received signal strength (RSS), time of flight (ToF), and angle of arrival (AoA). The earbud 114b may return an indication of the determined distance to earbud 114a via the communications interface 120c. More generally, technique may be used to determine distances between the earbuds 114a and 114b in a given earbud pair 102. Once determined, the distances between the earbuds 114a, 114b (as well as between two or more microphone arrays 208, as these distances are fixed) can be used as points in space to determine the position a sound originated, e.g., using triangulation, trilateration, beamforming, single or multiple microphone acoustic impedance measurements, or impulse/frequency response function measurements, etc.

Because the microphone arrays 208 are placed at known locations, the therapy application 214 and/or the models 216 may use these (or other) algorithms to calculate the exact position of the source of the sound. Doing so may allow the therapy application 214 to detect therapy devices being worn or otherwise used by the patient, detect pathologies, detect airway obstructions, etc. As described herein, the distances between the earbuds 114a, 114b (and the corresponding microphone arrays 208) may be periodically determined, e.g., to facilitate the detection of a pathology using both earbuds 114a, 114b, determination of a sleep position of the patient, detection of a device such as RPT device 106, mask 108, or other therapy devices 122 used by the patient, etc. Furthermore, using the models 216, the therapy application 214 and/or the models 216 may adjust the position determination algorithm to compensate for how sounds travel through the airway, how sounds travel through the ear canal, how sounds travel through fluid, how sounds travel through tissue, etc. Doing so allows the therapy application 214 and/or the models 216 to accurately determine the location where a sound originated.

In some embodiments, the therapy application 214 may detect the location of an airway obstruction in the patient based on sounds generated as the airway closes. In some embodiments, the therapy application 214 may determine a degree of the obstruction, e.g., partial, complete, etc. In some embodiments, the therapy application 214 may determine a type of the obstruction based on the sounds, e.g., OSA, positional sleep apnea, etc. In some embodiments, positional sleep apnea is determined based on position information received from the accelerometer 212 and/or one or more other wearables 110, e.g., to determine whether the patient is sleeping on their back, etc.

In some embodiments the therapy application 214 and/or the models 216 may determine whether the determined location is within the body of the patient, e.g., to identify external sounds. For example, the therapy application 214 and/or the models 216 may determine whether at least a portion of the sound was captured by the microphone arrays 208 through the body, e.g., through the airways, through the ear canal, through the tissues of the body, etc. If the therapy application 214 and/or the models 216 determine the sound was captured by the microphone arrays 208 through the body, the therapy application 214 and/or the models 216 may determine that the sound originated from within the body. In such an example, the therapy application 214/or the models 216 may exclude external therapy devices such as RPT device 106 as being the source of the sound and instead consider internal therapy devices as the source of the sound. Similarly, if the therapy application 214 and/or the models 216 may determine that the sound originated external to the body, the therapy application 214 may exclude internal therapy devices as being the source of the sound, and instead consider external devices such as RPT device 106 as the source of the sound.

In addition and/or alternatively, the therapy application 214 and/or the models 216 may consider the distance to the determined sound location relative to one or more of the earbuds 114a, 114b. For example, if the distance between the determined sound location and one or more of the earbuds 114a, 114b is 10 meters, the therapy application 214 and/or the models 216 may determine that the sound did not originate from within the body, as this distance is too great to originate from within the body. In addition and/or alternatively, the therapy application 214 and/or the models 216 may consider the direction of the sound captured by the microphone arrays 208. For example, if the direction of the sound indicates the sound was generated above and behind the earbuds 114a, 114b, the therapy application 214 and/or the models 216 may determine that the sound did not originate from within the body. The accelerometer 212 may provide position information to facilitate the direction from which the sound originated relative to the body, e.g., the head and/or ears of the body. Doing so may allow the therapy application 214 to determine specific therapy devices worn or otherwise used by the patient. For example, based at least in part on a determination that a sound originated near the patient's face, the therapy application 214 may determine the sound is associated with a mask 108 being worn by the patient. As another example, based at least in part on a determination that the sound originated a meter away from the patient, the therapy application 214 may determine the sound is associated with an RPT device 106 used to deliver therapy to the patient. Embodiments are not limited in these contexts.

More generally, the therapy application 214 and/or the models 216 may analyze one or more of the sounds detected by the plurality of microphone arrays 208. For example, the analysis may be used to determine a sleep position of the patient, determine a therapy device worn by the patient, determine a pathology associated with the sounds and/or device, determine errors or other problems with therapy devices, etc. For example, the therapy application 214 and/or the models 216 may perform waveform analysis on the soundwaves detected by the microphone arrays 208. For example, the therapy application 214 may compare the soundwaves (e.g., the waveforms, attributes of the soundwaves, etc.,) to known examples of types of sounds, e.g., sounds associated with devices, sounds associated with pathologies, etc. In some embodiments, the known types of sounds and associated sleep positions, pathologies, and/or associated devices may be stored in the therapy application 214, e.g., in the device profiles 224. As another example, the sounds associated with a particular sleep position may be stored in the user profiles 222. Therefore, if a sound (e.g., the sound of an airway obstruction) is similar to a stored sound associated with back sleeping in the user profile 222 of the person, the therapy application 214 may determine the person is sleeping on their back at least partially based on the sound.

As another example, if a sound is similar to a stored sound in the device profile 224 associated an RPT device 106, the therapy application 214 and/or the models 216 may determine the patient is using an RPT device 106, which may be associated with OSA. As such, the therapy application 214 and/or models 216 may determine the patient has OSA. As another example, if a sound is similar to a stored sound associated with a therapy device such as RPT device 106, mask 108, or other therapy device 122, the therapy application 214 and/or models 216 may determine that the patient is wearing or otherwise using the therapy device.

As stated, in some embodiments, the therapy application 214 determines a sleep position, device, a pathology, a location of the pathology, and/or any attribute thereof based at least in part on the models 216. The models 216 represent any type of model, such as a machine learning (ML) model, neural network, large language model (LLM), or any other type of artificial intelligence (AI) model. For example, the models 216 may include models trained to identify locations of input sounds, models trained to identify sounds based on input sounds (e.g., sounds associated with particular sleep positions, sounds of obstructions at a plurality of points in the airway, sounds of types of obstructions, sounds associated with therapy devices, sounds generated by other parts of the human body, etc.), models of the airway, models of the ear canal, models trained to determine how sounds travel through the airway, models trained to determine how sounds travel through the ear canal, models trained to determine how sound travels through tissues, models trained to determine how sounds travel through fluid, and models trained to generate treatments or other recommendations for identified sleep positions, pathologies, etc.

In artificial intelligence embodiments, the models 216 may be trained based on training data, e.g., data describing different sounds (and/or soundwaves) associated with sleep positions, data from a plurality of users, data describing therapies for pathologies, etc. For example, the training data may include sounds (and/or soundwaves), attributes of the sounds and/or soundwaves, etc. The models 216 may be trained to identify features of the sounds such that once trained, the models 216 may return a sound similar to an input sound. For example, the models 216 may be trained to identify the sounds of tissues collapsing in the airway. Based on an input sound of tissues collapsing in the airway, the models 216 may determine that the input sound is similar to the sound of tissues collapsing in the airway. Similarly, the models 216 may be trained to identify other data associated with input sounds, such as associated sleep positions, pathologies, therapy devices, treatments, etc. Therefore, the models 216 may return a sleep position associated with the input sound, a pathology associated with the sound, a therapy associated with the pathology, etc. The models 216 may be retrained over time, e.g., to be tailored to a particular user, sleep position, device, and/or pathology. Embodiments are not limited in these contexts.

As stated, the therapy application 214 and/or models 216 may consider other information to detect a sleep position, device, pathology, and/or a location thereof. For example, because the earbuds 114a, 114b are worn in the ear and the accelerometer 212 can provide orientation information, the therapy application 214 and/or models 216 are able to distinguish sounds coming from the airway, from within the ear, external to the body, etc. Therefore, the therapy application 214 and/or models 216 are able to filter out sounds originating from outside the body, etc., when determining a particular sleep position based on sounds. Furthermore, the therapy application 214 and/or models 216 may filter or otherwise ignore signals originating from within the body, but are not associated with sleep positions, therapy devices, and/or pathologies. For example, some digestion-related sounds may be identified and/or filtered (e.g., as not being associated with a sleep position, a device, and/or a pathology).

In some embodiments, the earbuds 114a, 114b may analyze the geometry of the ear canal. For example, the models 216 and/or user profiles 222 may include one or more models of geometry the human ear canal. The models of the geometry of the human ear canal may include models under various conditions, e.g., particular sleep positions, complete airway obstructions, partial airway obstructions, snoring, coughing, the presence of fluid in the ear canal, using a device (e.g., RPT device 106, mask 108, other therapy devices 122), using a device according to different configurations (e.g., using a mandibular advancement device in a number of different positions), etc. The models of the geometry of the ear canal may include models specific to a patient, and generic models that are not specific to any patients.

To determine a sleep position based on the geometry of the ear canal, the earbuds 114a, 114b may emit one or more vibrations and/or one or more sounds into the ear canal for acoustic reflectometry analysis. As these sounds and/or vibrations travel through the ear canal, some of the waves are reflected back towards the source (e.g., the earbuds 114a, 114b). The microphone arrays 208 and/or haptic feedback modules 210 may detect the reflected waves. The therapy application 214 may use acoustic reflectometry to create a model of the ear canal by analyzing the reflections of the emitted waves to map the geometry of the ear canal, thereby creating a model of the geometry of the ear canal. For example, the timing and intensity of the reflections provide information about the ear canal's shape and size. For example, the therapy application 214 may determine, based on the reflections, the acoustic impedance at different points along the ear canal. The acoustic impedance may be influenced by changes in the cross-sectional area and the presence of any blockages or abnormalities. The therapy application 214 and/or the models 216 may then use an algorithm to create the model of the ear canal. The generated model may then be used to detect differences, e.g., based on models of ear canal geometry stored by the therapy application 214, based on models of ear canal geometry stored by the models 216, previous models of the patient's ear canal generated and stored by the therapy application 214, etc.

For example, the therapy application 214 may compare the model of the ear canal created by the therapy application 214 to one or more stored models of the ear canal stored by the therapy application 214, e.g., in the user profiles 222 or the models 216. The therapy application 214 may determine a stored model that is similar to the input model based on the geometries. For example, if the input model is similar to a stored model of the ear canal while sleeping on their back, the therapy application 214 may determine the patient is sleeping on their back. As another example, the model created by the therapy application 214 may be provided as input to the models 216, which may return an indication of a pathology. For example, the features of the model of the ear canal may be similar to the features of an ear canal during a complete airway collapse by the models 216. Therefore, the models 216 may return an indication that the patient is experiencing the complete airway collapse. Based on the determination of the airway collapse and that the person is sleeping on their back, the therapy application 214 may generate a therapy recommendation.

As another example, the therapy application 214 may compare the model of the ear canal created by the therapy application 214 to one or more stored models of the ear canal stored by the therapy application 214, e.g., in the user profiles 222 and/or models 216. The therapy application 214 may determine a stored model that is similar to the input model based on the geometries. For example, if the input model is similar to a stored model of the patient's ear canal in the user profiles 222 (which may be associated with using an RPT device 106), the therapy application 214 may determine the patient is using an RPT device 106. For example, the features of the model of the ear canal may be similar to the features of a model of an ear canal during use of the RPT device 106 in the models 216 and/or user profiles 222.

In some embodiments, the earbuds 114a, 114b may operate according to two or more detection modes. For example, a first mode may include the earbuds 114a, 114b monitoring and analyzing sounds as described above. In such an example, a second mode may include the earbuds 114a, 114b periodically emitting sounds into the ear canal via the speaker 206 and/or vibrations into the ear canal via the haptic feedback module 210. The microphone arrays 208 and/or haptic feedback module 210 may detect response signals (e.g., reflections) from these sounds and/or vibrations. The therapy application 214 may then use triangulation, beamforming, trilateration, single or multiple microphone acoustic impedance measurements, impulse/frequency response function measurements, or any suitable location detection algorithm to detect a sleep position, a therapy device, an airway obstruction, pathology, or any attribute thereof. In some embodiments, the therapy application 214 determines a type and severity of an obstruction based at least in part on the vibrations. A third mode of operation may include using the geometry of the human ear canal to detect pathologies. A fourth mode of operation may include using the geometry of the human ear canal to detect therapy devices and/or errors or other associated problems with the devices. A fifth mode of operation may include detecting the sleep position of a person. A sixth mode of operation may include any combination of the first, second, third, fourth, and fifth modes of operation.

In some embodiments, the therapy application 214 may cause the earbuds 114a, 114b to change between the different modes of operation. The therapy application 214 may change the modes of operations according to any number and type of criteria. For example, the therapy application 214 may change the mode of operation at predetermined time intervals. In addition and/or alternatively, the therapy application 214 may change the mode of operation based on attributes of the waveform of a detected sound (e.g., pressure, amplitude, wavelength, frequency, etc.). For example, if the amplitude of a sound detected by the microphone arrays 208 exceeds a threshold amplitude associated with snoring, the therapy application 214 may change the mode of operation of the earbuds 114a, 114b, e.g., to determine the sleep position and cause the person to change their sleep position. As another example, the amplitude of a detected sound may be associated with airflow through the tube (or conduit) of a mask 108, and the therapy application 214 may determine the specific type of mask based on the amplitude of the detected sound. As another example, if the therapy application 214 detects a change in the geometry of the ear canal, the therapy application 214 may change the mode of operation to cause the earbuds to listen for sounds associated with pathologies and detect the location of the sounds as described herein. As another example, if the therapy application 214 detects a change in the geometry of the ear canal, the therapy application 214 may change the mode of operation to cause the earbuds to listen for sounds associated with sleep positions, determine the particular sleep position, and generate therapy recommendations as described herein. Embodiments are not limited in these contexts.

In some embodiments, the external devices 104, RPT devices 106, other wearables 110 and/or masks 108 include instances of the therapy application 214, not pictured in FIG. 1 for the sake of clarity. In such embodiments, the instances of the therapy application 214 on the earbuds 114a-114b communicate with the instances of the therapy application 214 on the external devices 104, RPT devices 106, other wearables 110, and/or masks 108 (e.g., to transmit notifications, recommendations, tasks, goals, or any other type of content). In some embodiments, the therapy applications 214 may transmit notifications via an operating system (not pictured) of the external devices 104, RPT devices 106, other wearables 110 and/or masks 108.

FIG. 3 shows a system including a patient 301 wearing a patient interface 303, in the form of nasal pillows, receiving a supply of air at positive pressure from an RPT device 106. The patient interface 303 represents the mask 108. Air from the RPT device 106 is humidified in a humidifier 305, and passes along an air circuit 304 to the patient 301. A bed partner 302 is also shown. The patient interface 303 is one example of a patient interface, or mask 108. Other examples include, but are not limited to, a nasal mask, a full-face mask, etc. As shown, the patient 301 is wearing earbud 114a (and corresponding earbud 114b, which is not depicted). In one or more embodiments, the earbuds, patient interface 303, RPT device 106, and humidifier 305 form a respiratory therapy system for treating a respiratory disorder.

As shown, the patient 301 is wearing a chest monitor 310, which may include heartrate monitoring capabilities among other biometric tracking capabilities. Similarly, the patient 301 is wearing a smartwatch 312, which may include various biometric tracking capabilities, e.g., heartrate tracking, oxygen saturation tracking using a pulse oximeter, stress tracking, sleep tracking, etc. The chest monitor 310 and smartwatch 312 represent the other wearables 110. Therefore, the chest monitor 310 and smartwatch 312 each include a respective processor 116b, memory 118b, communications interface 120b, and accelerometer 124.

As stated, the therapy application 214 of the earbuds 114a, 114b may determine the sleep position of the patient 301. For example, the accelerometer 212 of earbud 114b may provide acceleration data used by the therapy application 214 to determine the patient 301 is sleeping on their back. The therapy application 214 may further determine the angle at which the patient 301 is sleeping relative to the bed. Doing so may allow the therapy application 214 to provide a therapy recommendation based on the determined sleep position and/or angle.

As stated, the therapy application 214 may further consider other wearables 110 when determining the sleep position of the patient 301. For example, based on an analysis of the data from the smartwatch 312 (e.g., raw data from the accelerometer 124 and/or orientation information determined by the smartwatch 312 based on the raw data from the accelerometer 124), the therapy application 214 may determine the orientation of the smartwatch 312 is consistent with the back sleeping position. For example, if, for the smartwatch 312, the sensor axis pointing upward shows a dominant gravitational pull (e.g., the Z-axis is approximately 9.8 m/s², and the X and Y axes are approximately zero), the therapy application 214 may determine the smartwatch 312 (and therefore the wrist of the patient 301) are facing upward, which is consistent with a back sleeping position (as a −9.8 m/s² Z-axis may indicate the smartwatch 312 (and/or wrist) is facing down towards to the bed, which may be consistent with a stomach sleeping position).

In addition and/or alternatively, the therapy application 214 may consider the chest monitor 310 when determining the sleep position of the patient 301. For example, based on an analysis of the data from the chest monitor 310 (e.g., raw data from the accelerometer 124 and/or orientation information determined by the chest monitor 310 based on the raw data from the accelerometer 124), the therapy application 214 may determine the orientation of the chest monitor 310 is consistent with the back sleeping position. For example, if, for the chest monitor 310, the sensor axis pointing upward shows a dominant gravitational pull (e.g., the Z-axis is approximately 9.8 m/s², and the X and Y axes are approximately zero), the therapy application 214 may determine the chest monitor 310 (and therefore the chest or torso of the patient 301) are facing upward, which is consistent with a back sleeping position (as a −9.8 m/s² Z-axis may indicate the chest monitor 310 (and/or torso) is facing down towards to the bed, which may be consistent with a stomach sleeping position).

Therefore, based on the data from the smartwatch 312, the therapy application 214 may determine the orientation of the wrist, and based on the data from the chest monitor 310, the therapy application 214 may determine the orientation of the torso. Therefore, the multimodal sleep position of the patient 301 may reflect the orientation and/or position of the head (based on the data from the earbud 114b and/or 114a), the torso, and the wrist. Based on any one or more of these positions and/or orientations, the therapy application 214 may generate a therapy recommendation.

As stated, the therapy application 214 may consider additional factors when generating therapy recommendations for positional sleep therapy. For example, as shown, a soundwave 306 and/or a soundwave 308 may be detected by the microphone arrays 208 of the earbud 114a and/or 114b. The location the soundwaves 306, 308 originated may be determined by the therapy application 214 as described herein.

For example, the therapy application 214 may generally determine the soundwave 306 originated at a distance from the head of the patient 301. Similarly, the therapy application 214 may analyze the soundwave 306 and determine that the soundwave 306 is associated with sounds made by the RPT device 106. Therefore, the therapy application 214 may determine the patient 301 is using the RPT device 106. Based on the determination that the patient 301 is using the RPT device 106, the therapy application 214 may determine a pathology of the patient, e.g., that the patient 301 has OSA, POSA, CSA, etc. In some embodiments, the therapy application 214 may generate a therapy recommendation based on the sleep position of the patient 301, the use of the RPT device 106, and/or the determined pathology.

Furthermore, the therapy application 214 may generally determine the soundwave 308 originated close to the face of the person. Similarly, the therapy application 214 may analyze the soundwave 308 and determine that the soundwave 308 is associated with an airflow obstruction in the air circuit 304. The therapy application 214 may determine that the airflow obstruction is caused by the sleep position of the patient 301, e.g., the face and/or other parts of the body may be impinging the air circuit 304. Because the obstruction in the air circuit 304 may result in a reduction (and/or cessation) of the therapy delivered to the patient 301, the therapy application 214 may generate a therapy recommendation, e.g., generate a noise via the speakers 206 of the earbuds 114a, earbud 114b, to wake the patient 301 such that the sleep position is changed and blockage in air circuit 304 is cleared. Embodiments are not limited in these contexts.

FIG. 4 is a schematic 400 depicting the human auditory system. As shown, an earbud 114a is worn in on the right ear of a patient. Earbud 114b is pictured without picturing the left ear for the sake of clarity.

As shown, one or more sounds associated with one or more soundwaves 402 are emitted from within the body of the patient. The microphone arrays 208 of the earbud 114a and/or earbud 114b may detect the soundwaves 402 and provide data associated with the detection of the soundwaves 402 to the therapy application 214. At least a portion of the soundwaves 402 are received by the microphone arrays 208 via the ear canal 404. The therapy application 214 may determine a location of a source of the soundwaves 402 as described herein.

The therapy application 214 may determine a sleep position of the patient based at least in part on the soundwave 402. For example, the therapy application 214 may compare the soundwave 402 and/or features thereof to one or more stored sounds (e.g., in the user profiles 222 and/or models 216). The therapy application 214 may then determine one or more stored sounds that match or are otherwise similar to the stored sounds. For example, if the soundwave 402 matches or is otherwise similar to a stored sound associated with a supine sleep position in the user profile 222 for the patient, the therapy application 214 may determine the patient is sleeping in the supine position. As another example, a machine learning model or other AI model in the models 216 may receive features of the soundwave 402 as input and output a sleep position (e.g., prone, supine, side, etc.). In some embodiments, the AI model in the models 216 may further determine the sleep position based on the data from the accelerometers 212.

In some embodiments, therapy application 214 may generate a model of the ear canal 404 based at least in part on the soundwave 402. The therapy application 214 may analyze the model of the ear canal 404 to detect a change in the geometry of the ear canal 404, e.g., by comparing the model of the ear canal 404 to one or more stored models of the ear canal 404 in the user profiles 222. For example, the change may be based on the total volume of the ear canal 404, the shape of the ear canal 404, distances between two or more locations of the ear canal, etc.

In some embodiments, based on detecting the change in the ear canal 404, the therapy application 214 may determine the patient is in a particular sleep position. In some embodiments, the therapy application 214 may further consider the oxygen saturation values captured by the pulse oximeter 220 associated with each model was generated to determine the patient is wearing or otherwise using a therapy device.

As stated, in some embodiments, the therapy application 214 may process the soundwave 402 to determine the therapy device is being used. For example, if the soundwave 402 matches or is otherwise similar to the stored sound of a therapy device in the device profiles 224, the therapy application 214 may determine the specific type of therapy device being used by the patient. Similarly, by detecting pathologies associated with the patient (e.g., in the device profiles 224 and/or user profiles 222), the therapy application 214 may further generate positional sleep therapy recommendations based on the determined pathologies and/or devices.

For example, the therapy application 214 may determine that the pathology is an airway obstruction, and may identify other attributes of the airway obstruction (e.g., the type of the obstruction, whether the obstruction is partial, complete, etc.). The therapy application 214 may then generate a recommended a treatment or therapy 218 for the type of obstruction and the determined sleep position of the patient. For example, the therapy application 214 may determine to modify one or more operating parameters of the RPT device 106 to cause the RPT device 106 to deliver therapy to the patient. In such embodiments, the therapy application 214 may transmit an instruction to the RPT device 106 to modify the one or more operating parameters, thereby improving the therapy provided to the patient. The therapy application 214 may further transmit an indication of the detected device, pathology, therapy 218, and/or any attributes thereof to other devices in the system 100.

FIG. 5 is a schematic 500 illustrating an example of using earbuds to analyze sounds, in accordance with one embodiment. FIG. 5, which is not to scale, depicts a patient 502 wearing earbuds 114a, 114b. As shown, earbud 114a includes microphone arrays 208-1, 208-2, and 208-N, while earbud 114b includes microphone arrays 208-3, 208-4, and 208-M, where “N” and “M” are any positive integer (and “N” and “M” may be the same or different integers).

As shown, a soundwave 504 is emitted from the patient 502. Because the distances between each microphone array 208 in each earbud 114a, 114b, are known (e.g., fixed), each earbud 114a, 114b can independently determine the location of the origin of the soundwave 504, e.g., using triangulation, trilateration, single or multiple microphone acoustic impedance measurements, impulse/frequency response function measurements, and/or beamforming. For example, the soundwave 504 and each microphone array 208 may be a point in space to facilitate the generation of triangles to compute the location the soundwave 504 originated.

Similarly, the earbuds 114a, 114b may collectively determine the location of the origin of the soundwave 504. For example, earbud 114b may provide information describing the soundwave 504 detected by microphone arrays 208-3, 208-4, and 208-M to the earbud 114a. The therapy application 214 may then use the information from the microphone arrays 208 of both earbuds 114a, 114b to compute the location of the origin of the soundwave 504.

Furthermore, as described above, the therapy application 214 may use the determined location and any attributes of the soundwave 504 to determine a sleep position of the patient 502. For example, the therapy application 214 may compare the soundwave 504 and/or features thereof to one or more stored sounds (e.g., in the user profiles 222). The therapy application 214 may then determine one or more stored sounds that match or are otherwise similar to the stored sounds. For example, if the soundwave 504 matches or is otherwise similar to a stored sound associated with a prone sleep position in the user profile 222 for the patient, the therapy application 214 may determine the patient is sleeping in the prone position. As another example, a machine learning model or other AI model in the models 216 may receive features of the soundwave 504 as input and output a sleep position (e.g., prone, supine, side, etc.).

Furthermore, as described above, the therapy application 214 may use the determined sleep position to generate a therapy recommendation. In some embodiments, the therapy application 214 implements the recommendation, e.g., by causing the RPT device 106 to change one or more parameters of operation, etc. As another example, notifications or other instructions may be sent by the therapy application 214 to other devices in the system 100. Embodiments are not limited in these contexts.

Furthermore, as described above, the therapy application 214 may use the determined location and any attributes of the soundwave 504, the detected device, the patient 502, sleep position, the ear canal, and/or the airway to determine a pathology associated with the soundwave 504, e.g., an OSA event, etc. The therapy application 214 may further determine a therapy 218 associated with the determined pathology. In some embodiments, the therapy application 214 implements the therapy 218, e.g., by causing the RPT device 106 to change one or more parameters of operation, etc. Embodiments are not limited in these contexts.

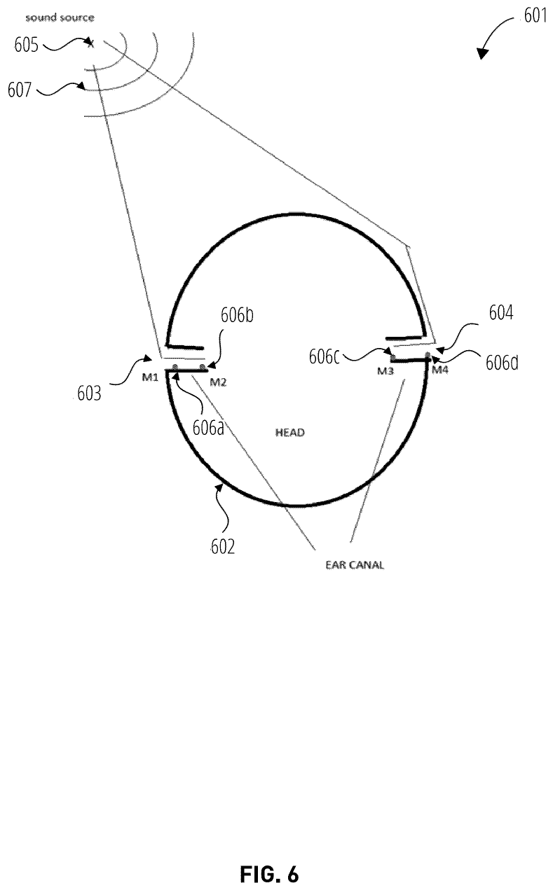

FIG. 6 is a schematic 601 illustrating an example of using earbuds to detect sleep position, therapy devices, airway obstructions, and/or other pathologies, in accordance with one embodiment. FIG. 6, which is not to scale, depicts the ear canals 603, 604 of a human head 602. Furthermore, two microphones (or microphone arrays) of an earbud (not pictured) are depicted in the ear canal of the person. For example, microphones 606a and 606b (denoted by M1 and M2, respectively) may be included in a first earbud (not pictured) located at or near ear canal 603. Similarly microphones 606c and 606d (denoted by M3, and M4, respectively) may be included a second earbud (not pictured) located at or near ear canal 604. The microphones 606a-606d are representative of one or microphones and/or microphone arrays. For example, each microphone 606a-606d may be representative of a respective microphone array 208. In some embodiments, to determine the location of a sound emitted from a sound source 605, any two or more of the microphones 606a-606d may define a microphone array. Embodiments are not limited in these contexts.

As stated, the distances between any two of microphones 606a-606d may be known or otherwise determined. As shown, as one or more soundwaves 607 generated at sound source 605 moves through space, portions of the soundwaves 607 may enter each ear canal 603, 604. Therefore, portions of the soundwaves 607 may be detected by the microphones 606a-606d at different times. For example, microphone 606a may detect soundwaves 607 prior to the time microphone 606b detects soundwaves 607. Similarly, microphone 606d may detect soundwaves 607 prior to microphone 606c.

Therefore, the phase relationship between the soundwaves 607 detected at each microphone 606a-606d may be used to determine the location of the sound source 605. For example, the therapy application 214 of an earbud may determine the phase shift of soundwave 607 between microphone 606a and microphone 606b, while the therapy application 214 of the other earbud in the pair may determine the phase shift of soundwaves 607 between microphone 606c and microphone 606d. As another example, one earbud may determine the phase shift of soundwaves 607 between microphone 606a and microphone 606d. Embodiments are not limited in these contexts, as the phase shift may be determined between any two or more of the microphones 606a-606d. The therapy application 214 may then compare or otherwise use the phase shifts to triangulate the location of the sound source 605, e.g., using triangulation, trilateration, time difference of arrival (TDOA), etc. For example, by converting phase shifts to time differences, the TDOA between multiple (e.g., two or more of) microphones 606a-606d may be computed to determine the location of the sound source 605.

The therapy application 214 may determine that the soundwaves 607 are associated with a sleep position of the patient based at least in part on the determined location of the sound source 605. For example, by determining the sound source 605 is centrally located a few centimeters meters away from the earbuds 114a, 114b (e.g., in the patient's throat), the therapy application 214 may determine the patient is the sound source 605. Based on an analysis of the soundwaves 607, the therapy application 214 may determine the sound is associated with snoring, sleep apnea events, etc., e.g., a pathology. Based on detecting the pathology and location thereof, the therapy application 214 may generate a positional sleep therapy recommendation. For example, based on a determination that the person is sleeping in a supine position, the determined location of the soundwaves 607, and the detection of the pathology, the therapy application 214 may generate a therapy recommendation, e.g., to cause the patient to change sleep positions, modify parameters of the RPT device 106, etc. Embodiments are not limited in these contexts.

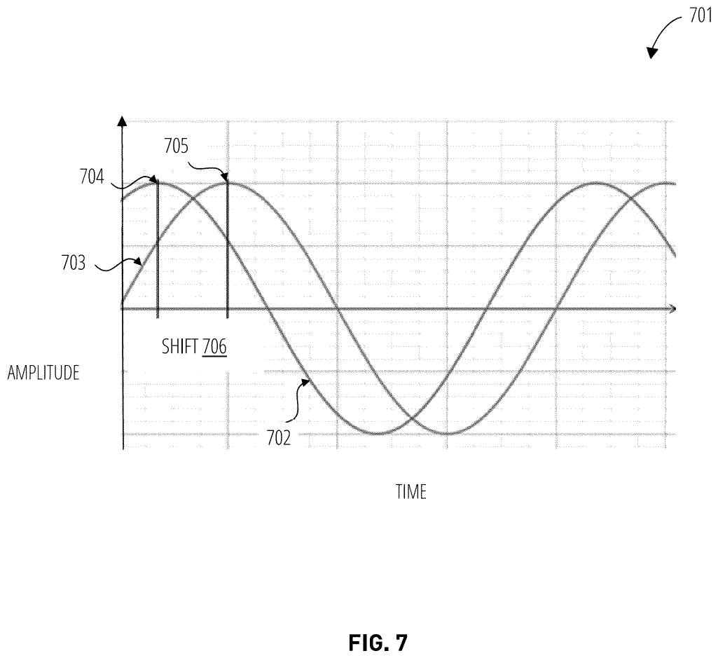

FIG. 7 is an example graph 701 depicting two waveforms of a sound, where the x-axis corresponds to time and the y-axis corresponds to amplitude. As shown, waveforms 702 and 703 are depicted in the graph 701. In one example, the waveform 702 may be detected by microphone 606a, while waveform 703 may be detected by microphone 606b. Although the waveforms 702 and 703 appear similar, they are shifted due to the different times the sound is detected by a respective microphone, which is based on the distance between the microphones and the speed of sound. Therefore, the phase shift 706 of waveform 702, 703 may be determined according to any suitable technique. For example, determining the phase shift at two points 704, 705 may be based on cross-correlation which measures the similarity between waveforms 702, 703 as a function of the time-lag applied to one of them. For example, the cross-correlation may be computed using a Fast Fourier Transform (FFT). As another example, the time difference between points 704, 705 may be computed. The cross-correlation and/or time difference may be a time shift. The time shift may be used to compute phase shift of the waveforms 702, 703.

As another example, the therapy application 214 may compute phase shift 706 by comparing the phases of waveforms 702, 703 in the frequency domain. For example, the therapy application 214 may compute the Fourier Transform of both waveforms 702, 703 to determine their frequency components. The therapy application 214 may identify the phases of the frequency components and compute the phase shift based on computing the difference of the identified phases of the frequency components. More generally, once the time shift and/or phase shifts are determined, the location a sound originated may be determined. For example, using the phase and/or time shift between microphones 606a, 606b, and 606c may be used to determine TDOA at each microphone, which may then be used to determine the location a sound originated. Embodiments are not limited in these contexts.

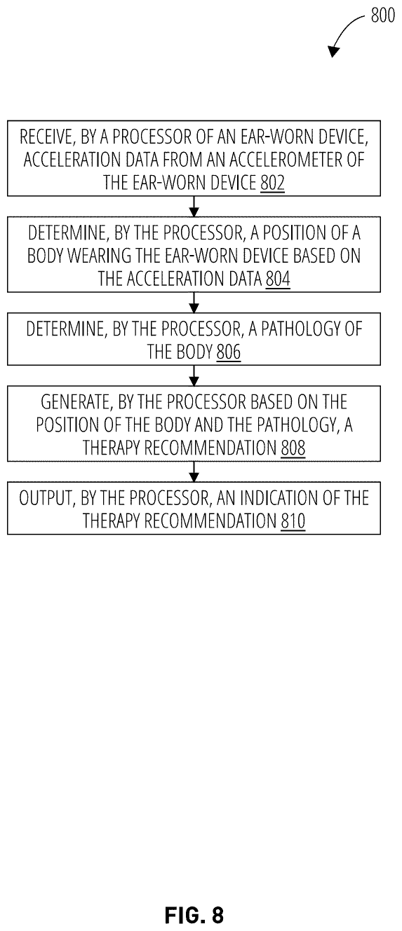

FIG. 8 illustrates an example logic flow 800 for using ear-worn devices to provide positional sleep therapy. Although the example logic flow 800 depicts a particular sequence of operations, the sequence may be altered without departing from the scope of the present disclosure. For example, some of the operations depicted may be performed in parallel or in a different sequence that does not materially affect the function of the logic flow 800. In other examples, different components of an example device or system that implements the logic flow 800 may perform functions at substantially the same time or in a specific sequence. Furthermore, in some embodiments, the logic flow 800 may be used in combination with other techniques and/or logic flows to determine a sleep position of a patient and/or provide a therapy recommendation.

According to some examples, the logic flow 800 includes receiving, by a processor of an ear-worn device, acceleration data from an accelerometer of the earbud at block 802. For example, the processor 116e of earbud 114a may receive acceleration data from accelerometer 212 of the earbud 114a.

According to some examples, the logic flow 800 includes determining, by the processor, a position of a body wearing the ear-worn device based on the acceleration data at block 804. For example, the processor 116e may determine position of a body wearing the earbud 114a based on the acceleration data. The position may be a sleep position and may include positions of different parts of the body.

According to some examples, the logic flow 800 includes determining, by the processor, a pathology of the body at block 806. For example, the processor 116e may determine a pathology of the body. The pathology may be determined based on any suitable technique, such as referencing indications of pathologies in the user profile 222, detecting the pathology using soundwave analysis, detecting therapy devices such as RPT device 106, mask 108, etc.

According to some examples, the logic flow 800 includes generating, by the processor based on the position of the body and the pathology, a therapy recommendation at block 808. For example, the processor 116e may generate, based on the position of the body and the pathology, a therapy recommendation. For example, the therapy recommendation may be to change sleep positions, adjust the operating parameters of the RPT device 106, etc.

According to some examples, the logic flow 800 includes outputting, by the processor, an indication of the therapy recommendation at block 810. For example, the processor 116c may output an indication of the therapy recommendation. For example, the speakers 206 may output sounds to wake the patient, the haptic feedback modules 210 may generate vibrations to wake the patient, etc. As other examples, the indication of the therapy recommendation may be transmitted to other devices via the communications interface 120e, e.g., to adjust one or more parameters of the RPT device 106, order a new type of mask 108, send the recommendation to a medical provider's computing device, output the recommendation on the user's smartphone or smartwatch, etc.

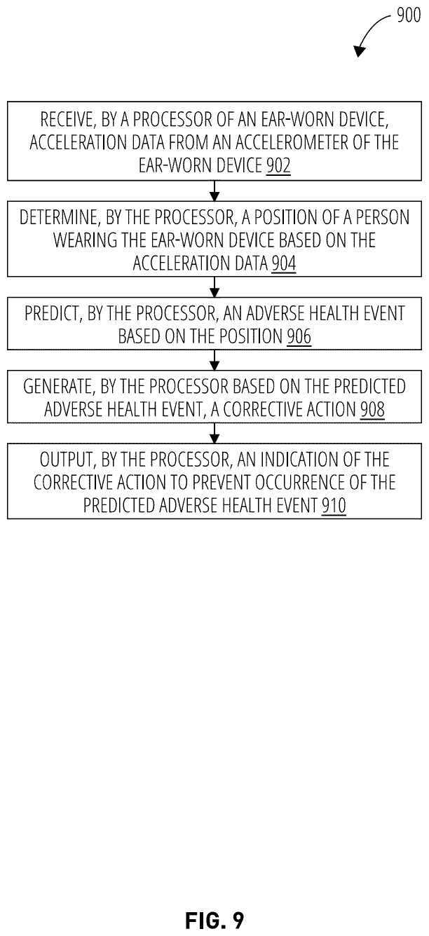

FIG. 9 illustrates an example logic flow 900 for predicting adverse health events using ear-worn devices such as earbuds. Although the example logic flow 900 depicts a particular sequence of operations, the sequence may be altered without departing from the scope of the present disclosure. For example, some of the operations depicted may be performed in parallel or in a different sequence that does not materially affect the function of the logic flow 900. In other examples, different components of an example device or system that implements the logic flow 900 may perform functions at substantially the same time or in a specific sequence.

According to some examples, the logic flow 900 includes receiving, by a processor of an ear-worn device, acceleration data from an accelerometer of the ear-worn device at block 902. For example, the processor 116e of earbud 114a may receive acceleration data from an accelerometer 212 of the earbud 114a.

According to some examples, the logic flow 900 includes determining, by the processor, a position of a body wearing the ear-worn device based on the acceleration data at block 904. For example, the processor 116e may determine a position of a person wearing the ear-worn device based on the acceleration data. For example, based on the acceleration data, the processor 116e may determine the person is sleeping on their back.

According to some examples, the logic flow 900 includes predicting, by the processor, an adverse health event based on the position of the body at block 906. For example, the processor 116e may predict an adverse health event based on the position of the body. For example, the processor 116e may predict that the person, who is sleeping on their back, may have an apnea or a hypopnea.

According to some examples, the logic flow 900 includes generating, by the processor based on the predicted adverse health event, a corrective action at block 908. For example, the processor 116e may generate, based on the predicted adverse health event, a corrective action to prevent the occurrence of the adverse health event. Example corrective actions include notifying the person, modifying therapy provided by an RPT device 106, etc.

According to some examples, the logic flow 900 includes outputting, by the processor, an indication of the corrective action to prevent occurrence of the predicted adverse health event at block 910. For example, the processor 116e may output, an indication of the corrective action to prevent occurrence of the predicted adverse health event. Example indications include outputting sounds via the speakers 206, emitting vibrations via the haptic feedback modules 210, outputting notifications on the user's smartphone and/or smartwatch, transmitting an instruction to cause the RPT device 106 to modify therapy, etc. Embodiments are not limited in these contexts.

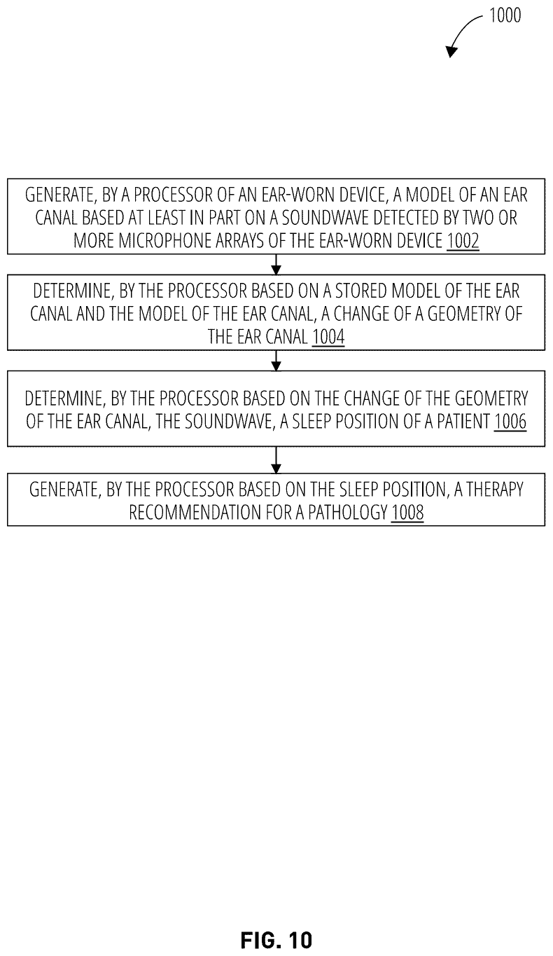

FIG. 10 illustrates an example logic flow 1000 for using ear-worn devices such as earbuds to determine a sleep position of a patient. Although the example logic flow 1000 depicts a particular sequence of operations, the sequence may be altered without departing from the scope of the present disclosure. For example, some of the operations depicted may be performed in parallel or in a different sequence that does not materially affect the function of the logic flow 1000. In other examples, different components of an example device or system that implements the logic flow 1000 may perform functions at substantially the same time or in a specific sequence. Furthermore, in some embodiments, the logic flow 1000 may be used in combination with other techniques and/or logic flows to determine a sleep position of a patient and/or provide a therapy recommendation.

According to some examples, the logic flow 1000 includes generating, by a processor of an ear-worn device, a model of an ear canal based at least in part on a soundwave detected by two or more microphone arrays of the ear-worn device at block 1002. For example, the therapy application 214 executing on earbud 114a illustrated in FIG. 1 may generate a model of an ear canal based at least in part on a soundwave detected by two or more microphone arrays 208 of the earbud 114a.

According to some examples, the logic flow 1000 includes determining, by the processor based on a stored model of the ear canal and the model of the ear canal, a change of a geometry of the ear canal at block 1004. For example, the therapy application 214 executing on earbud 114a may determine, based on a stored model of the ear canal stored in the user profile 222 of the patient and the model of the ear canal generated at block 1002, a change of a geometry of the ear canal. For example, the change may be based on the volume of the ear canal, distances between two points in the ear canal, etc.

According to some examples, the logic flow 1000 includes determining, by the processor based on the change of the geometry of the ear canal and the soundwave, a sleep position of a patient at block 1006. For example, the therapy application 214 executing on earbud 114a may determine, based on the change of the geometry of the ear canal determined at block 1004, that the patient is in a particular sleep position (e.g., supine, prone, side, or any combination thereof). For example, the change in geometry may indicate the patient is experiencing poor respiration (which may be associated with sleeping on the back) and/or may have characteristics similar to a stored model of the ear canal (e.g., in the user profile 222) that is associated the user while sleeping on the back. Similarly, the soundwave may have one or more characteristics that are similar to one or more characteristics of stored sounds in the user profiles 222, e.g., known sounds of the patient while sleeping on the back. Therefore, the therapy application 214 may determine the patient is sleeping on their back.

According to some examples, the logic flow 1000 includes generating, by the processor based on the respiratory therapy device, a therapy recommendation at block 1008. For example, the therapy application 214 executing on earbud 114a may generate, based on the sleep position, a therapy recommendation. The therapy recommendation may be any type of recommendation. For example, the therapy recommendation may include an audible alert and/or vibrations to cause the patient to change sleep positions, prescribing a new and/or modified treatment, ordering a new therapy device, ordering replacement parts for the therapy device, waking the patient, etc. An indication of the therapy recommendation may be transmitted and/or outputted via one or more of the earbuds 114a-114b, the external devices 104, other wearables 110, masks 108, and/or RPT devices 106. Embodiments are not limited in these contexts.

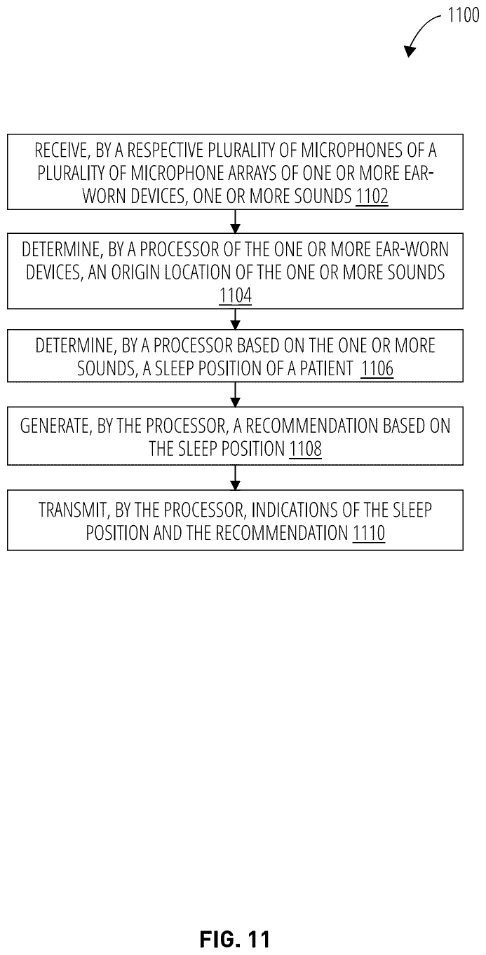

FIG. 11 illustrates an embodiment of a logic flow 1100. The logic flow 1100 represents some or all of the operations executed by one or more embodiments described herein. For example, the logic flow 1100 includes some or all of the operations performed by devices or entities in the system 100 to use ear-worn devices such as earbuds to determine a sleep position of a patient based at least in part on soundwave detection. Embodiments are not limited in these contexts. Furthermore, in some embodiments, the logic flow 1100 may be used in combination with other techniques and/or logic flows to determine a sleep position of a patient and/or provide a therapy recommendation.

In block 1102, logic flow 1100 receives, by a respective plurality of microphones of a plurality of microphone arrays 208 of one or more ear-worn devices such as earbuds 114a, 114b, one or more sounds. The sounds may include ambient sounds, sounds generated by devices in the system 100, sounds generated by the body of the wearer of the ear-worn devices, etc. In block 1104, logic flow 1100 determines, by a processor 116e of the one or more ear-worn devices, a location the one or more sounds originated from. For example, the therapy application 214 and/or the models 216 of earbuds 114a, 114b may process one or more of the sounds and determine the location based on triangulation, trilateration, beamforming, single or multiple microphone acoustic impedance measurements, impulse/frequency response function measurements, or any suitable technique.

In block 1106, logic flow 1100 determines, by the processor based on the one or more sounds, a sleep position of a patient. For example, the therapy application 214 and/or the models 216 of earbuds 114a, 114b may process one or more of the sounds and determine the sleep position based on the sounds, e.g., by identifying similar sounds in the user profile 222 associated a with particular sleep position. For example, the sound may be a snoring sound and may match a sound in the user profile 222 that is associated with the patient sleeping on their stomach. In block 1108, logic flow 1100 generates, by the processor based on the sounds and sleep position, a recommendation based on the sleep position determined at block 1106. For example, the therapy application 214 of earbuds 114a, 114b may determine to modify an of the therapy provided by the RPT device 106 to the patient, e.g., increasing pressure, flow, etc.

At block 1110, the processor transmits an indication of the recommendation to a device via a network. For example, the therapy application 214 of earbuds 114a, 114b may instruct the RPT device 106 to change an attribute of the therapy delivered to the wearer. In response, the RPT device 106 may change the attribute, which may increase the oxygen saturation of the patient's blood. Embodiments are not limited in these contexts.

In some embodiments, a system 1204 may be provided for measuring physiological parameters, e.g., characteristics of a patient while they are awake. The system may be configured to provide a patient 1203 with ongoing monitoring of their waking physiological parameters, e.g., an “awake state” to determine, e.g., quantify, whether their sleep quality improves as a result of respiratory therapy, e.g., PAP therapy. In this regard, the system 1204 may be considered a patient tracker 1204 configured to measure a patient's physiological state during the day, rather than at night.

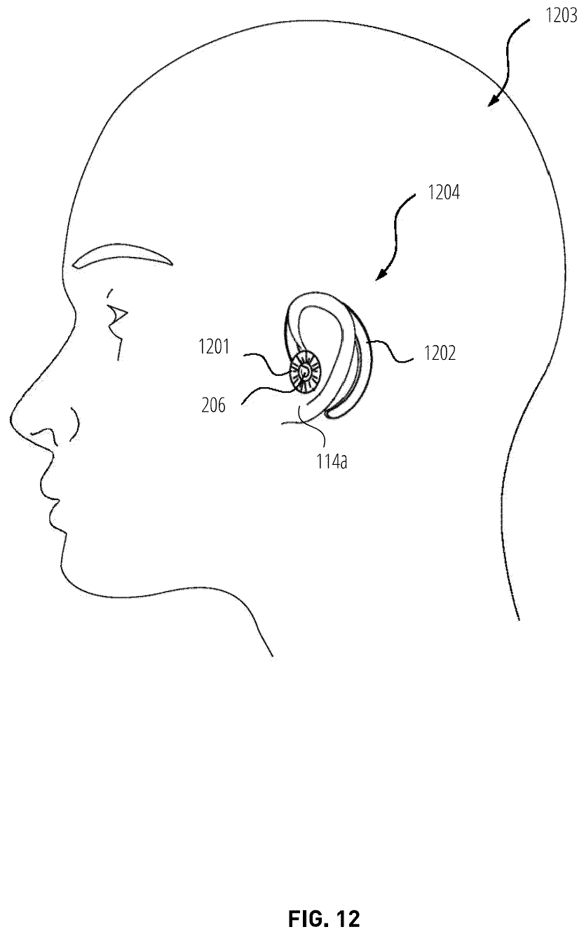

Referring to FIG. 12, the system 1204 may be implemented in the form of a wearable device, such as the “earbud” type wearable device shown wearable with respect to a person's ear canal, ear lobe or behind the person's ear. In this regard, the patient tracker 1204 may be considered a patient tracking device 1204 (also referred to as device 1204). Therefore, in at least one embodiment, the device 1204 is the earbud 114a and/or earbud 114b.

The patient tracking device 1204 may be configured to measure daytime activities and physiological characteristics, e.g., conditions, of the patient. For example, the system may be configured to measure activities such as: a distance travelled by the patient; a number of steps (or paces) walked, run, climbed, etc.; a type, duration, intensity, etc., of physical activity; time spent standing.

The daytime activities set forth above may influence the physiological characteristics of the patient. For example, a patient travelling a distance may have an elevated heart rate, increased breath rate, etc. The physiological characteristics that can be measured by the system include: a respiration rate, variability of respiration, etc.; a heart rate, variability of heart rate, etc.; a magnitude of calories burned; blood oxygen saturation; electrodermal activity (e.g., skin conductance or galvanic skin response); or any combination thereof.

Referring to FIG. 12, the device 1204 may be used by itself, or independently of, e.g., without a the RPT device 106. In particular, the device is shown without a patient interface 1301. In this form, the patient may, for example, wear the device by itself during daytime activities such as walking, running, etc.

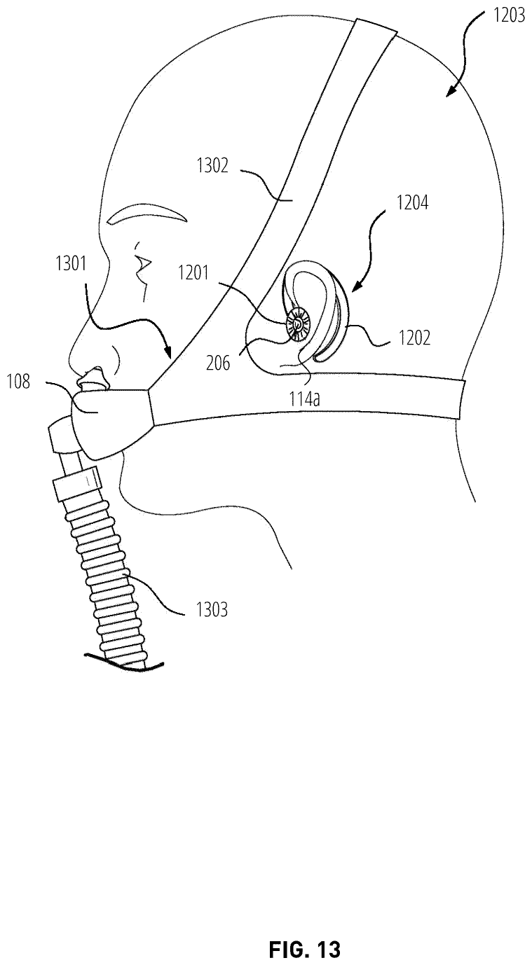

By comparison, and as shown in FIG. 13, the device 1204 may also be used together with an RPT device such as RPT device 106. In the form shown, the device is worn together with a patient interface 1301 (as part of the RPT device 106). The patient may wear the device in this way, e.g., with the patient interface 1301, while they sleep for recording data while also receiving respiratory therapy.

As shown in FIG. 12 and FIG. 13, the device 1204 comprises a body 1201 for housing the control system, memory device, sensors, batteries (rechargeable or replaceable), etc. An ear hook 1202 is provided for locating, e.g., attaching, mounting, etc., the device 1204 about the patient's ear. In particular, the ear hook is configured, e.g., shaped, to locate and removably secure behind the patient's external ear (e.g., auricle/pinna).

The body 1201 is configured to locate within the patient's ear for transmitting audio (e.g., sound) into the ear for the patient to hear. At least a portion of the body 1201 may be configured to releasably secure within at least a portion of the patient's external auditory canal. In this regard, the body of the device may be shaped similar to a traditional earbud used for transmitting audio into a patient's ear.

The patient tracker 1204 may be configured to receive the physiological data about the patient from the one or more sensors (e.g., sensors 204, not pictured for clarity). In some forms, the patient tracker may also be configured to receive environmental data from the one or more sensors.

The environmental data may relate to environmental conditions surrounding the patient (e.g., the environmental data being related to the patient), such as temperature, humidity, etc. In either form of data, e.g., physiological or environmental, the data may be stored in the memory device and analyzed by the processor(s) of the control system.

Advantageously, measuring and recording data relating to the environmental conditions surrounding the patient may allow the device 1204 to accommodate for environmental conditions that influence the physiological conditions of the patient. For example, if the humidity and temperature of air surrounding the patient is high, the patient may fatigue more rapidly when e.g., walking, than in colder, less humid conditions. In effect, environmental conditions (such as high humidity and temperature) may inadvertently indicate the patient is fatigued as a result of e.g., a lack of sleep. Hence, allowing the device 1204 to accommodate for such environmental conditions means that indications of the patient's sleep quality can be more accurately presented to the patient.

For example, an optical sensor using red, infrared, and/or green, could be used to calculate a photoplethsmogram. Subsequently, parameters such as pulse rate (PR), pulse rate variability (PRV), SpO₂ can be determined. If respective sensors are placed on a periphery of the user, e.g., at their skin, the peripheral arterial tone may also be measured.

Generally, the types of sensors 204 utilized in the patient tracker 1204 may vary according to the physiological and/or environmental data being generated. For example, when the patient tracker 1204 is integrated into an item of clothing, it may comprise the electromyography (EMG) sensor for detecting electrical signals generated by muscles. Alternatively, the EMG sensor may not be utilized when the patient tracker is integrated into a ring. In any case, each of the one or more sensors may be configured to output sensor data that is received and stored in the memory device of the patient tracker 1204.

In some forms of the device 1204, one or more of the sensors set forth above may be configured to contact the patient's skin. In this regard, the sensors may be located on an externally facing surface of the body 1201 or the ear hook 1202, so as to be in contact with the patient's skin when in use. This allows, e.g., the galvanic skin response (GSR) sensor, to measure changes in sweat gland activity on the skin. In another example, the one or more sensors, e.g., optical sensor, may be located on the ear hook so as to contact an area of skin between the patient's auricle/pinna and hairline.

Other forms of the sensors may be configured for mounting internally to the body 1201 and ear hook 1202, such as the motion sensor. In this case, for example, the motion sensor may be integrated within the body of the device and configured to measure a patient's head movement.

As set forth above, the one or more sensors of the patient tracker 1204 can be configured to determine an awake state of the patient. The patient tracker utilizes the physiological and environmental data generated from the sensors to determine how “awake,” e.g., alert, the patient is for a duration of non-sleep, e.g., during the daytime. For example, the device 1204 may be configured to measure a patient's heart rate and EEG during the daytime. Based on the physiological data generated from variations in the heart rate and EEG measurements, the system 1204 may indicate how awake the patient is, e.g., if the patient is lethargic and has an unfocussed attention during the daytime.

In order to determine an awake state, including stages of a sleep (such as NREM (N1, N2, N3/SWS) or REM), data may be fed into an artificial Intelligence (AI) or Machine Learning (ML) model such as the one or more of the models 216. This model 216 may be trained on the IMU and PPG signals, or pre-processed parameters of those.

Breathing and/or respiration signal related parameters can include: variability of breathing rate throughout the day and/or night (the variability being characteristic of the person)—this can be inter-breath or over longer timescales—e.g., 30, 60, 90 sec or much longer periods; the stability over time (related to the variability); the standard deviation of breathing rate; the depth of respiration (shallow, deep etc.), and relative amplitude of adjacent breaths; the mean or average value of the breathing rate; the trimmed mean (e.g., at 10%) to reject outliers; wake or Asleep (e.g., the detected sleep stage of the person); surges (sudden accelerations or decelerations) in breathing rate seen during quiet periods and during REM sleep; median (50th percentile); interquartile range (25th-75th percentile); 5th-95th percentile; 10th-90th percentile; shape of histogram; skewness; kurtosis; peak frequency over time; ratio of second and third harmonics of peak frequency; percentage of valid data (Valid Physiologically Plausible Data); autocorrelation of the individual signals; characteristic patterns in the spectrogram; wake or asleep; relative percentage of REM and deep sleep.

Cardiac signals can be processed to produce features such as: heart rate variability HRV (inter beat (e.g., as derived from the Ballistocardiogram) and over longer defined moving windows—e.g., 30, 60, 90 sec); variability over time (interbeat/breath variability); mean; trimmed mean (10%); standard deviation; median (50th percentile); interquartile range (25th-75th percentile); 5th-95th percentile; 10th-90th percentile; shape of histogram; skewness; kurtosis; stability over time; peak frequency over time; ratio of second and third harmonics of peak frequency; percentage of valid data (Valid Physiologically Plausible Data), wake or asleep; autocorrelation of the individual signals; characteristic patterns in the spectrogram.

Cardiorespiratory signals can be formed, such as: magnitude square cross spectral density (in a moving window); cross coherence; respiratory sinus arrhythmia peak; low frequency (LF)/high frequency (HF) ratio to indicate autonomic nervous system parasympathetic/sympathetic balance (LF is often defined as around 0.04-0.15 Hz, whereas HF is around 0.15-0.4 Hz); the cross correlation; cross coherence (or cross spectral density) of the heart and breathing signal estimates; non-linear estimates such as entropy measures; the characteristic movement patterns over longer time scales, e.g., the statistical behavior observed in the signals; patterns of movement during detection of and comparison of these heart and breathing signals (e.g., during sleep, some people may have more restful and some more restless sleep).

Based on the determination of a patient's awake state, the patient tracker 1204 may provide the patient with an indication of how effective their respiratory therapy, e.g., PAP therapy, is at improving their sleep quality. For example, in the case where physiological data indicates the patient is lethargic and unfocussed, such an indication may be correlated with a low efficacy of the patient's PAP therapy.

Conversely, in the case where physiological data indicates the patient has improved capacity for daytime activities, e.g., a lower resting heart rate, etc., such an indication may be correlated with a high efficacy of the patient's PAP therapy. As set forth in more detail later, the patient tracker may be configured to alert the patient of such indications, e.g., notifications that e.g., a morning run, positively impacted their sleep.

The patient tracker 1204 may be configured to measure an efficacy of respiratory therapy by recording a baseline measure of “off therapy” physiological data and comparing this to an “on therapy” measure of physiological data. According to the changes detected in the measured data, the patient tracker may advise the patient of either improvements to their sleep performance, or deteriorations to their sleep performance.

In a variation, the patient may be advised of improvements that occur in their ability to undertake daytime activities, such as capacity for exercise, that are a result of their corresponding improvements to their sleep performance. Conversely, the patient tracker can be configured to notify the patient of a deteriorated capacity to perform daytime activities as a result of a corresponding deterioration in their sleep performance. Advantageously, notifying a patient of said changes to either their sleep performance or capacity for daytime activities can allow a patient to understand an impact of their respiratory therapy.

As part of providing the patient with an indication of how effective their respiratory therapy is, the patient tracker 1204 may also be configured to record, e.g., “timestamp” events associated with the patient's sleep periods. For example, the various sensors of the patient tracker 1204 may be configured to record a time that the patient wakes after a period of sleep, times when the patient wakes during a period of sleep (e.g., a rate of sleep disturbances), a time that the patient exits the bed, a time that the patient enter the bed, etc. These events may be utilized, e.g., analyzed, together with other sensor data gathered about the patient, to determine how awake the patient may be as a result of their e.g., PAP therapy.

Advantageously, data relating to e.g., when a patient wakes, may be longitudinally recorded so as to determine sleeping patterns of the patient. This information may be processed and utilized to inform the patient of e.g., whether they are ready for sleep; whether they are sleeping well; whether they should expect to feel tired during their waking hours, etc. Ultimately, the patient tracker 1204 may provide the patient with an indication of how effective their respiratory therapy has been.

Set forth below are some further examples of sensors such as sensors 204 that may be used with the patient device 1204, and their application for use with the patient device 1204.

In some forms of the patient tracker 1204 where the motion sensor is utilized (as set forth previously), the motion sensor may generate data relating to specific movements of the patient, such as exercise (e.g., running), or other body (e.g., limb) movements. These movements may be utilized to determine the patient's awake state. For example, a patient's limb movements may be analyzed and determined as being slow relative to a standard measurement of the patient's normal movements.

While the motion sensor is described in broad terms, the motion sensor may be specifically one or more inertial sensors, such as accelerometers, gyroscopes, and magnetometers. These types of motion sensors may be selected, e.g., utilized according to their optimal use-case.

In some forms of the motion sensors, the motion sensors may be configured to detect motion or acceleration associated with arterial pulses, such as pulses in or around the face of the patient and in particular, those proximal to the patient tracking device 1204, e.g., the body 1201. The motion sensors in this form may be configured to detect features of the pulse shape, speed, amplitude, or volume that may be analyzed to indicate qualities of a patient's awake state.

In other forms, an EEG sensor may also be provided in the patient tracker 1204 for measuring physiological data relating to the patient's brain. The EEG sensor may include one or more dry electrodes positioned on or around the scalp of the patient. In this form, the EEG may locate within, or extend from, a portion of the ear hook 1202 or body 1201. For this reason, the EEG sensor is optimally utilized when the patient tracker is implemented as an earpiece, as shown in FIG. 12 and FIG. 13, such that the external surfaces of the body 1201 and ear hook 1202 may be in contact with the patient's scalp.

Depending on the placement of the EEG sensors, it may be possible to detect EEG slowing during the daytime (such as a higher ratio of delta and theta frequencies to alpha and beta frequencies) and relate this daytime slowing to greater daytime sleepiness. Thus, it may be possible to avoid asking a patient if they have daytime sleepiness, but rather, derive it from EEG slowing vs. a baseline and relate this to a reduced movement of the patient (detected from e.g., an accelerometer such as accelerometer 212).

In forms where a PPG sensor is provided to measure, e.g., a heart rate, the patient tracker is optimally configured to contact the patient's skin. In this form, the patient tracker may be integrated into a piece of clothing to optimally generate data relating to e.g., a heart rate pattern, a heart rate variability, a cardiac cycle, respiration rate, estimated blood pressure, or any combination thereof.

When the patient tracker is integrated into an earbud and/or earpiece as shown in FIG. 12 and FIG. 13, a speaker 206 may be provided for outputting (e.g., generating) audio. The audio, e.g., generated sounds, are configured to be projected into the patient's ear so as to be heard by the patient. For example, the patient tracker 1204 may be configured as a type of earphone to play music for a patient to listen to during the day. In another example, the patient tracker 1204 may be configured to sound an alarm for waking the patient from sleep, reminding them of an event (e.g., a calendar event). In yet a further example, the device 1204 may assist in relaxation of the patient prior to sleep by playing controlled breathing audio cues. In yet a further example again, the device 1204 may also provide hearing assistance, whereby the device may be coupled with a smartphone to generate audio, amplify audio, etc.

In some implementations, the speaker 206 may be used together with, or substituted by, a bone conduction speaker. In this form, the bone conduction speaker is not configured to generate audio for the patient to hear via their ears, rather, the speaker generates vibrations that are configured to penetrate the patient's temporal bones. In variations, an audio and bone conduction speaker may be configured for use together.

In some further implementations, the speaker 206 may be a noise cancelling speaker for assisting in reduction of background noises. Advantageously, this may be used prior to sleep, for reducing background noises that may otherwise hinder sleep.

In either form of the speaker, e.g., as a speaker, bone conduction speaker, noise cancelling speaker, etc., the patient tracking device 1204 may be coupled (e.g., wired or wirelessly) to a computing device such as external device 104, e.g., a mobile phone, for playing music or otherwise generating the sounds for the patient to hear. In the case of a wireless connection, the patient tracking device 1204 may be configured to communicate through various communication protocols, such as, Wi-Fi, Bluetooth, etc. The patient tracking device may thereby include an antenna, a receiver, a transmitter, a transceiver, or any combination thereof for communicating with wirelessly with a computing device.

The external device 104 may be configured to operate, e.g., run software configured to communicate with the patient tracking device 1204. In forms where the external device 104 is a mobile phone or tablet, the software may be configured as a mobile application allowing the patient to control operation of the patient tracking device via the mobile device.

The external device 104 may be used as a way to display information about the patient's awake state or other data in the user profiles 222. In other forms, the computing device may also be configured to process (via one or more processors) data generated from the patient tracking device 1204. In further forms, the external device 104 may be configured to receive input from the patient for controlling operation of the patient tracking device. As set forth above, the input of the patient may relate to the patient configuring the patient tracking device 1204 to send diary alarms, or in other cases, to select music to listen to (via the speakers).

In some forms of the patient tracking device, the patient may input information into the computing device, e.g., via the software, for determining, at least in part, the awake state of the patient. That is, the patient may self-report information that may not be sensed, per se, but be provided by the patient to be considered together with physiological and/or environmental data generated by the sensors. The combination of self-reported data and sensed data may be analyzed to determine a patient's awake state.

The self-reported information input by the patient may include demographic information, biometric information, therapy device use, medical information such as medications, etc., diet(s), subjective stress level of the patient, subjective fatigue level of the patient, subjective health status of the patient, a recent life event experienced by the patient, or any combination thereof. In the case of the medical information, the patient may provide information relating to one or more medical conditions, medication usage, etc.

Referring to FIG. 13, the patient tracking device 1204 (e.g., earbud 114a or 114b) may be configured for use with respiratory therapy, e.g., an RPT device such as RPT device 106. The RPT device may include the patient interface 1301, a conduit 1303, a mask 108 and a positioning and stabilizing structure 1302. It is noted that although a particular mask is shown in FIG. 13, other types of masks may be utilized, such as a full-face mask, nasal mask, oro-nasal mask, etc.

As set forth above, the patient tracker 1204 may provide the patient with ongoing monitoring of their “awake” state and provide feedback to the patient regarding any differences detected between “on” and “off” therapy. In other words, the patient tracker 1204 may indicate changes in the patient's sleep performance after they begin respiratory therapy and, in effect, indicate to the patient how effective their use of respiratory therapy has been.

The patient tracker 1204 may be configured to correlate changes in a patient's daytime activities with their adherence and/or compliance to e.g., CPAP therapy. For example, in patients having symptoms such as chronic fatigue, daytime sleepiness, cognitive impairment, etc., the patient tracker (as set forth previously) may be configured to monitor for improvements in such symptoms. The patient tracker may be configured to determine correlations between these improvements, e.g., changes, and the patient's adherence and/or compliance to CPAP therapy. These correlations may be reported, e.g., communicated as feedback to the patient, so that the patient is aware of the positive impact their adherence and/or compliance to CPAP therapy has on their capacity for performing daytime activities.

The patient tracker 1204 may be configured to interrelate a specific respiratory therapy, e.g., CPAP and a deterioration of healthy behaviors or an improvement of healthy behaviors. That is, the patient tracker 1204 may also be configured to monitor and report to a patient their unhealthy behaviors which may occur as a result of sleep related breathing disorders.

For example, patients having sleep apnea for an extended period of time prior to diagnosis may develop unhealthy behaviors, such as lack of exercise, bad sleep habits, etc., which may persist even after commencing respiratory therapies, e.g., CPAP. The sensor(s) and self-reported information input into the patient tracker 1204 may be used to monitor and report to the patient such behaviors. Reporting these behaviors as feedback to the patient may assist the patient to change, e.g., re-train such behaviors. Advantageously, re-training the patient to remove said unhealthy behaviors can positively impact their respiratory therapy, in addition to reducing the patient's risk of comorbidities.

In this regard, the patient tracker 1204 can provide the patient with a complete treatment for their sleep related breathing disorder(s). That is, in addition to opening the patient's airways via, e.g., PAP therapy, the patient tracker can identify and treat unhealthy behaviors that are symptomatic of the sleep related breathing disorder. Advantageously, this can motivate a patient to be more adherent and/or compliant to respiratory therapy.

In some forms, the patient tracker 1204 may be configured to provide the patient with detailed correlations of their improved daytime activities and corresponding compliance to respiratory therapy. For example, the patient tracker 1204 may be configured to record use of therapy devices and correlate a patient's use of CPAP therapy during a sleep period, with the patient being able to run a larger distance the following day, or the patient having a lower resting heart rate, etc.

The patient tracker 1204, as set forth above, can be configured to improve a patient's adherence and/or compliance by behavioral intervention. That is, the patient tracker 1204 can be configured to allow a patient to break, e.g., intervene, particular habits that are associated with their sleep related breathing disorder(s).

In some forms, a patient's compliance and/or adherence to a respiratory therapy may also be detected by measurements taken by the one or more sensors of the patient tracker 1204. For example, the patient tracker may include an EEG configured to measure daytime markers of a patient's increased alertness. Such markers may be compared against normal measures of the patients' alertness, such that an indication of the patients' improved alertness can be determined. This can indicate an improved efficacy of the respiratory therapy, and in turn, indicate the patients' adherence and/or compliance to therapy. Advantageously, use of the sensors to automatically detect efficacy and therapy adherence and/or compliance means that the patient may not be required to monitor their perceived daytime sleepiness, e.g., lethargy or reduced alertness to determine an efficacy of their respiratory therapy.

Furthermore, utilizing the EEG for compliance indications may also allow for a detection of impaired cognitive function. That is, detection of a patient's alertness may be used as a proxy for an assessment of their cognitive function.

In some forms, the patient tracking device 1204 may be coupled with the RPT device 106 to monitor the patient's sleep stage during periods of sleep. In this form, the sensors of the patient tracking device 1204 may be used together with the sensors of the RPT device 106 (e.g., optionally located in the patient interface 1301, flow generator, or other component of the RPT device 106), for detecting e.g., states of a sleep cycle. In some embodiments, the data collected may be used to determine a sleep position of the patient. In some forms, the data collected may be used to inform the patient of how effective their respiratory therapy has been, and in other forms the data may be additionally or alternatively used to adjust the delivery of respiratory therapy, e.g., pressure, flow rate, etc. Therefore, the earbuds 114a, 114b, and/or the therapy application 214, may instruct the RPT device 106 to adjust pressure, flow rate, etc., based on a sleep position detected by the therapy application 214.

As stated, one or more microphone arrays 208 may also be provided to the patient tracking device 1204 to measure a patient's breathing during sleep. In this form, a microphone array 208 may be located proximal to the patient's mouth and/or nose, and so accurately record breathing sounds, e.g., in the user profiles 222. A detection of abnormal breathing may be indicative of a sleep apnea, whereby the patient tracking device (e.g., earbuds 114a-114b) may be used together with the RPT device 106 to adjust therapy, e.g., pressure, flow, etc., for stimulating a change in the patient's breathing.

In some further forms, one or more motion sensors 204 described previously may be utilized during a patient's sleeping period to detect movements of the patient. For example, a number of movements during a sleep period may be detected, and used to provide an indication of e.g., a disturbed sleep. In some forms, the data collected from the motion sensor may be fused, e.g., coupled, combined, integrated, etc., with flow data collected from the RPT device 106. This combination of data may be used to improve sleep and/or wake classification, e.g., determination of a patient's awake state.

In other forms, the patient tracker 1204 may be configured to monitor the patient's sleep stage without being coupled to the RPT device 106. In this form, the patient tracker 1204 may be configured to detect and record physiological and/or environmental data as it would when coupled with the RPT device 106. However, rather than adjust operation of the RPT device, the patient tracker 1204 in this form would utilize the data recorded to inform the patient of their sleep performance, e.g., apnea events, etc.

In some further forms where the patient tracking device 1204 is used without being coupled to the RPT device 106, the data collected during a sleeping period may be implemented as a change to respiratory therapy at a later date. That is, the data collected when the patient is not wearing the patient interface 1301 may be used to adjust therapy the next time the patient wears the patient interface 1301.

In some further forms, the patient tracking device 1204 may be configured to intermittently couple with the RPT device 106 so as to communicate with the RPT device. The patient tracking device 1204 in this form may be configured to operate both together with the RPT device 106, and independently of the RPT device 106. That is, when the patient is near the RPT device 106, the patient tracker 1204 may be able to connect (e.g., wirelessly) with the RPT device 106. When the patient is away from the RPT device 106, e.g., walking outside, the patient tracker may be able to operate independently of the RPT device 106.

For example, the patient tracker 1204 operating independently may be able to temporarily record and store data from the sensors for later communicating said data to the RPT device 106 when the patient tracker 1204 is proximal to the RPT device 106.

In this form, the patient tracking device 1204 may be worn together with the patient interface 1301 in some instances, e.g., during sleep, and in other instances the patient tracking device may not be worn with the patient interface 1301, e.g., when a patient leaves their home. In some cases, missing data e.g., data which is not collected by either the RPT device 106 or patient tracking device 1204, may be collected from an alternative data source, such as a wrist worn accelerometer or HR sensor. For example, the device 1204 may be coupled with an external device 104 such as a smart watch, or a smart hub for collecting data that may not be captured by the device 1204 or the RPT device 106.

In some forms, the external device 104, e.g., mobile device, as set forth previously may be configured to connect with the patient tracker 1204 when the patient tracker 1204 is not coupled with the RPT device 106. In this regard, the device 1204 may be configured to log and process data within its memory, without requiring a wireless connection for a period of time.

In some forms, the patient tracker 1204 may be utilized for detecting and diagnosing a patient with an un-treated sleep related breathing disorder. The patient tracker 1204 used in this form may allow a patient to determine whether they require respiratory therapy e.g., PAP therapy, positional therapy, insomnia treatment, etc. In this form, the sensors (as set forth previously) may be configured to register (e.g., detect) a sleep event that is indicative of a sleep related breathing disorder.

In forms whereby the patient tracker 1204 is configured for detecting and diagnosing a patient with a sleep related breathing disorder, the patient tracker 1204 may be utilized to monitor a patient's daytime activities to determine indications of sleep related breathing disorders. For example, a patient may develop unhealthy behaviors, such as lack of exercise, bad sleep habits, etc., that may be detected and utilized as an indicator of insomnia, etc.

In some embodiments, the patient tracker 1204 may be utilized for detecting a patient with an under-treated sleep related breathing disorder. That is, a patient having already been diagnosed with a sleep related breathing disorder, but is not receiving effective therapy. In this case, the patient tracker 1204 may be configured to monitor e.g., heart rate variability for indicating whether the patient is under-treated. In response, the patient tracker 1204 may be configured to provide the patient with an indication of how to adjust therapy during the night, or alternatively, the patient tracker may be configured to automatically adjust a respiratory therapy device (as set forth previously) to appropriately treat the under-treated disorder.

In some forms, the patient tracker 1204 can also be configured for detecting and monitoring for comorbidities of sleep apnea. For example, the sensor(s) set forth above may be configured for detecting diabetes, heart failure, stroke, and obesity.

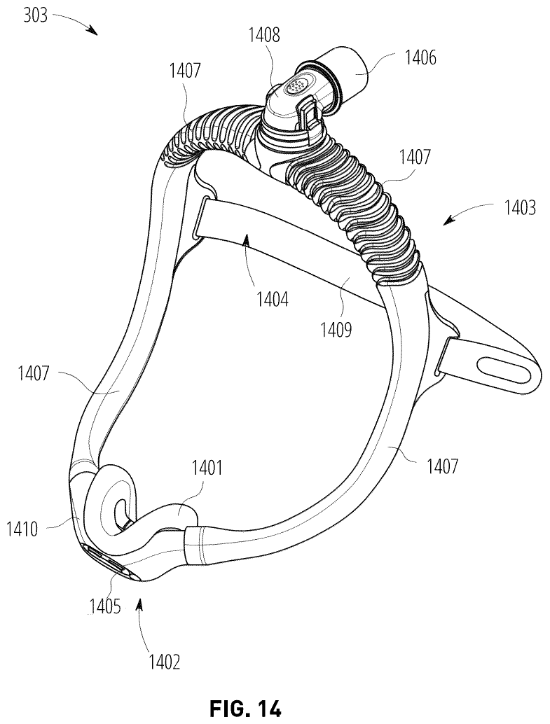

FIG. 14 shows a patient interface 303 having conduit headgear 1404, in accordance with one embodiment. The patient interface 303 is one example of the mask 108 of FIG. 1, and therefore includes a processor 116d, memory 118d, and communications interface 120d (not pictured for clarity).

As shown, a non-invasive patient interface 303 includes a seal-forming structure 1401, a plenum chamber 1402, a positioning and stabilizing structure 1403, a vent 1405, an elbow 1408, a strap 1409, a cushion module 1410, and one embodiment of connection port 1406 for connection to air circuit 304. In some forms a functional aspect may be provided by one or more physical components. In some forms, one physical component may provide one or more functional aspects. In use the seal-forming structure 1401 is arranged to surround an entrance to the airways of the patient so as to maintain positive pressure at the entrance(s) to the airways of the patient 301. The sealed patient interface 303 is therefore suitable for delivery of positive pressure therapy, e.g., in the form of supplementary gas 1537 (e.g., oxygen).

As stated, the patient interface 303 can communicate with other devices, such as the earbuds 114a-114b and the RPT device 106, e.g., to receive instructions and modify respiratory therapy provided via the patient interface based on the instructions. The patient interface 303 is constructed and arranged to be able to provide a supply of air at a positive pressure above the ambient, for example at least 2, 4, 6, 10, or 20 cmH2O with respect to ambient.

In some embodiments, the positioning and stabilizing structure 1403 comprise one or more headgear tubes 1407 that deliver pressurized air received from a conduit forming part of the air circuit 304 from the RPT device to the patient's airways, for example through the plenum chamber 1402 and seal-forming structure 1401. In the embodiment illustrated in FIG. 14, the positioning and stabilizing structure 1403 comprises two tubes 1407 that deliver air to the plenum chamber 1402 from the air circuit 304. The tubes 1407 are configured to position and stabilize the seal-forming structure 1401 of the patient interface 303 at the appropriate part of the patient's face (for example, the nose and/or mouth) in use. This allows the conduit of air circuit 304 providing the flow of pressurized air to connect to a connection port 1406 of the patient interface in a position other than in front of the patient's face, for example on top of the patient's head.

As shown, the patient interface 303 includes a vent 1405 constructed and arranged to allow for the washout of exhaled gases, e.g., carbon dioxide. In some embodiments, the vent 1405 is configured to allow a continuous vent flow from an interior of the plenum chamber 1402 to ambient whilst the pressure within the plenum chamber is positive with respect to ambient. The vent 1405 is configured such that the vent flow rate has a magnitude sufficient to reduce rebreathing of exhaled CO2 by the patient while maintaining the therapeutic pressure in the plenum chamber in use.

Connection port 1406 allows for connection to the air circuit 304. In one or more embodiments, the patient interface 303 includes a forehead support. In one or more embodiments, the patient interface 303 includes an anti-asphyxia valve.

Air may be delivered to the patient in one of two main ways. In one example, the patient may receive the flow of pressurized air through headgear tubes 1407. This may be referred to as a “tube up” configuration and may position a connection port at the top of the patient's head. In another example, the patient may receive the flow of pressurized air through a conduit connected to the plenum chamber 1402, for example through the connection port 1406. This may be referred to a “tube down” configuration where the airflow conduit is positioned in front of the patient's face.

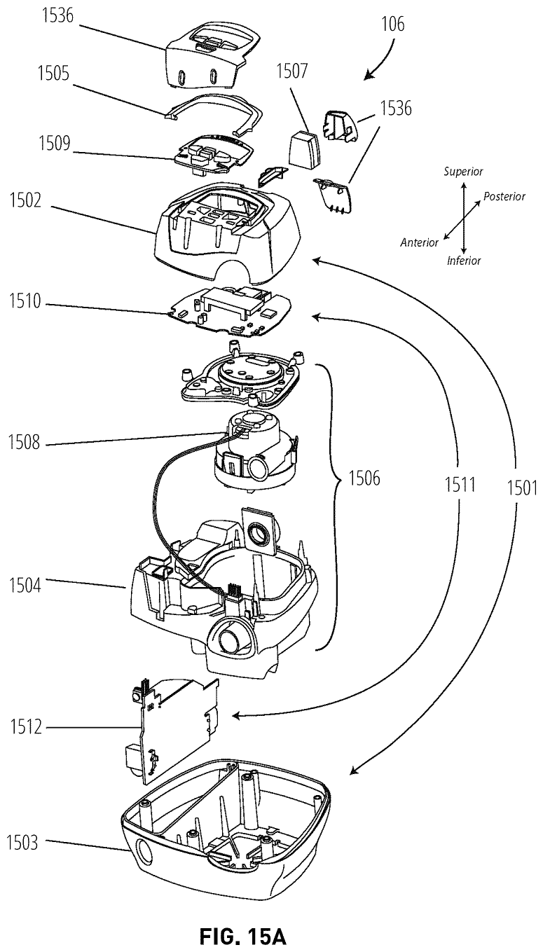

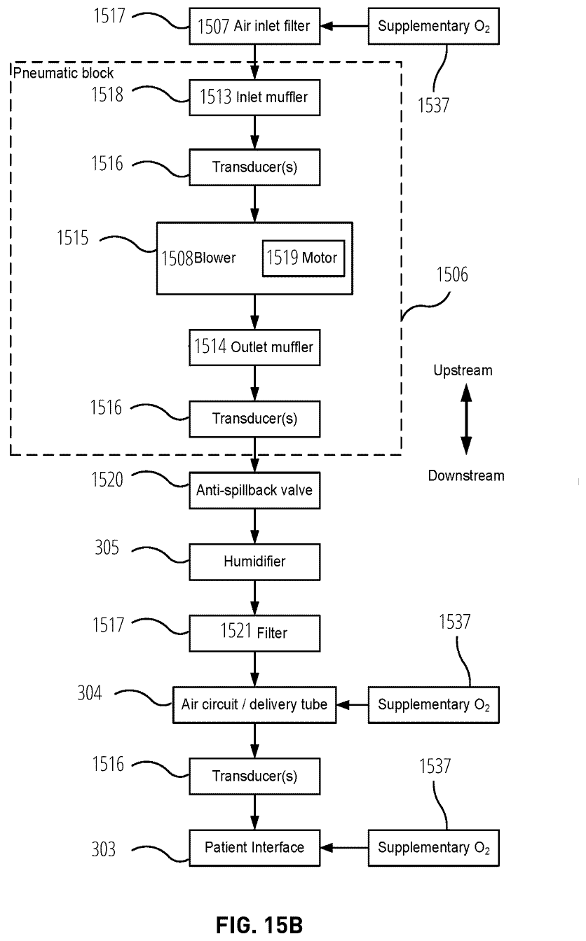

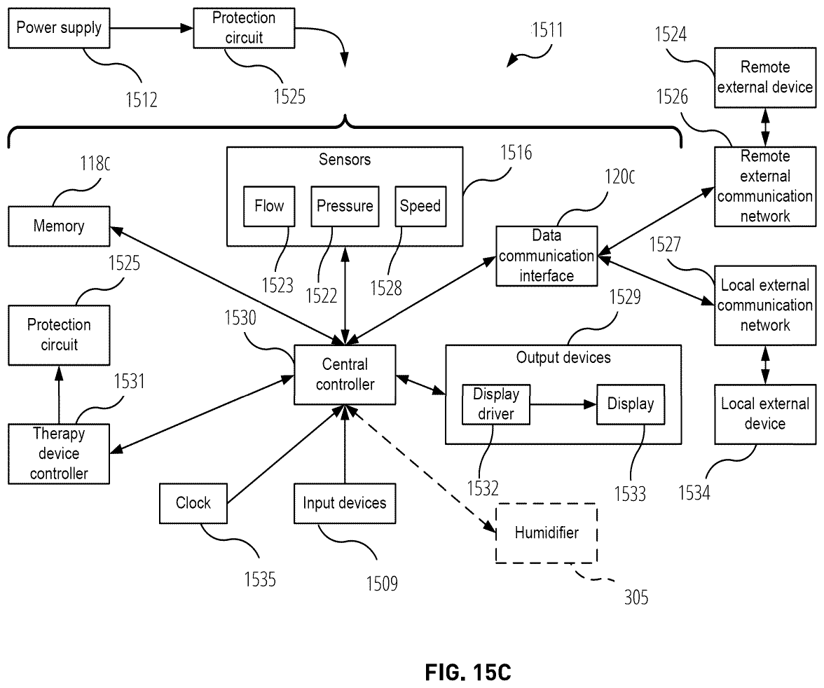

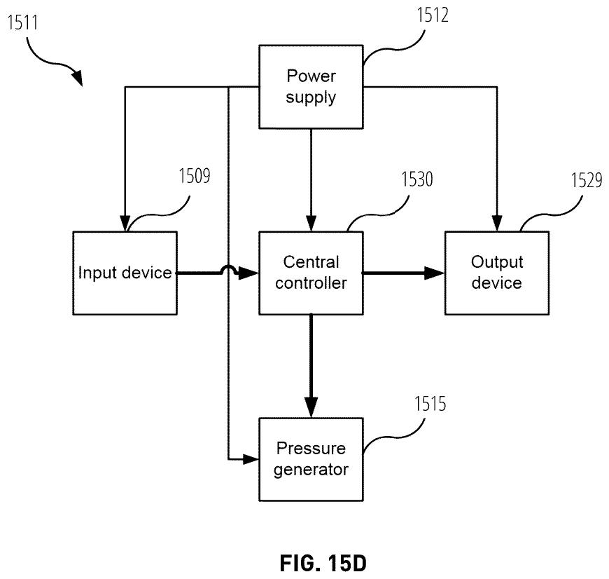

FIG. 15A shows an RPT device 106 in accordance with one embodiment. The RPT device 106 comprises mechanical, pneumatic, and/or electrical components and is configured to execute one or more algorithms, such as any of the methods, in whole or in part, described herein. The RPT device 106 may be configured to generate a flow of air for delivery to a patient's airways, such as to treat one or more of the respiratory conditions described elsewhere herein. For example, the therapy application 214 may cause the RPT device 106 to generate the flow of air to treat a pathology detected according to the techniques disclosed herein. Doing so may include the RPT device 106 modifying the delivery of therapy using one or more of the components depicted in FIG. 15A-FIG. 15D, which may include modifying any attribute thereof.

As shown in FIG. 15A, the RPT device 106 may have an external housing 1501, formed in two parts, an upper portion 1502 and a lower portion 1503. Furthermore, the external housing 1501 may include one or more panel(s) 1536. The RPT device 106 comprises a chassis 1504 that supports one or more internal components of the RPT device 106. The RPT device 106 may include a handle 1505.

One or more of the air path items may be located within a removable unitary structure which will be referred to as a pneumatic block 1506. The pneumatic block 1506 may be located within the external housing 1501. In one embodiment a pneumatic block 1506 is supported by, or formed as part of the chassis 1504.

FIG. 15B is a schematic diagram of the pneumatic path of an RPT device 106 in accordance with one or more embodiments. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

As shown in FIG. 15B, the pneumatic path of the RPT device 106 may comprise one or more air path items, e.g., an inlet air filter 1507, an inlet muffler 1513, a pressure generator 1515 capable of supplying air at positive pressure (e.g., a blower 1508), an outlet muffler 1514 and one or more transducers 1516, such as pressure sensors 1522 and flow rate sensors 1523.

The RPT device 106 may include an air filter 1517, or a plurality of air filters 1517. In the embodiment illustrated in FIG. 15B, an inlet air filter 1507 is located at the beginning of the pneumatic path upstream of a pressure generator 1515. In some embodiments, an outlet air filter 1521, for example an antibacterial filter, is located between an outlet of the pneumatic block 1506 and a patient interface 303. The RPT device 106 may include a muffler 1518, or a plurality of mufflers 1518. In one or more embodiments, an inlet muffler 1513 is located in the pneumatic path upstream of a pressure generator 1515. In one or more embodiments, an outlet muffler 1514 is located in the pneumatic path between the pressure generator 1515 and a patient interface 303.

In some embodiments, a pressure generator 1515 for producing a flow, or a supply, of air at positive pressure is a controllable blower 1508. For example, the blower 1508 may include a brushless DC motor 1519 with one or more impellers. The impellers may be located in a volute. The blower may be capable of delivering a supply of air, for example at a rate of up to about 120 liters/minute, at a positive pressure in a range from about 4 cmH2O to about 20 cmH2O, or in other forms up to about 30 cmH2O when delivering respiratory pressure therapy.

The pressure generator 1515 may be under the control of the therapy device controller 1531. In other forms, a pressure generator 1515 may be a piston-driven pump, a pressure regulator connected to a high pressure source (e.g., compressed air reservoir), or a bellows. The therapy device controller 1531 may receive instructions from the therapy application 214 and adjust the therapy based on the instruction.

In some embodiments, one or more transducers 1516 are located upstream and/or downstream of the pressure generator 1515. The one or more transducers 1516 may be constructed and arranged to generate signals representing properties of the flow of air such as a flow rate, a pressure or a temperature at that point in the pneumatic path.

In some embodiments, one or more transducers 1516 may be located proximate to the patient interface 303. In one or more embodiments, a signal from a transducer 1516 may be filtered, such as by low-pass, high-pass or band-pass filtering.

In some embodiments, a motor speed transducer 1528 is used to determine a rotational velocity of the motor 1519 and/or the blower 1508. A motor speed signal from the motor speed transducer 1528 may be provided to the therapy device controller 1531. The motor speed transducer 1528 may, for example, be a speed sensor, such as a Hall effect sensor.

As shown in FIG. 15B an anti-spill back valve 1520 is located between the humidifier 305 and the pneumatic block 1506. The anti-spill back valve is constructed and arranged to reduce the risk that water will flow upstream from the humidifier 305, for example to the motor 1519.

FIG. 15C is a schematic diagram of the electrical components 1511 of an RPT device such as RPT device 106 in accordance with one embodiment.

As shown in FIG. 15C, the RPT device 106 comprises an electrical power supply 1512, one or more input devices 1509, a central controller 1530, a therapy device controller 1531, a pressure generator 1515, one or more protection circuits 1525, memory 118c, transducers 1516, communications interface 120c and one or more output devices 1529. Electrical components 1511 may be mounted on a single Printed Circuit Board Assembly (PCBA) 1510. In an alternative form, the RPT device 106 may include more than one PCBA 1510.

The power supply 1512 may be located internal or external of the external housing 1501 of the RPT device 106. In one or more embodiments, power supply 1512 provides electrical power to the RPT device 106 only. In another embodiment, power supply 1512 provides electrical power to both RPT device 106 and humidifier 305.

In some embodiments, one or more flow rate sensors 1523 may be based on a differential pressure transducer. In one or more embodiments, a signal generated by the flow rate sensor 1523 and representing a flow rate is received by the central controller 1530. The RPT device 106 may include a clock 1535 that is connected to the central controller 1530.

In some embodiments, therapy device controller 1531 is a therapy control module that forms part of one or more algorithms executed by the central controller 1530. In one or more embodiments, therapy device controller 1531 is a dedicated motor control integrated circuit. The therapy device controller 1531 and the central controller 1530 represent the processor 116b of FIG. 1.

The one or more protection circuits 1525 may comprise an electrical protection circuit, a temperature and/or pressure safety circuit. Memory 118c may be located on the PCBA 1510. Memory 118c may be in any form. Additionally, or alternatively, RPT device 106 includes a removable form of memory 118c, for example a memory card made in accordance with the Secure Digital (SD) standard.

In one or more embodiments, the communications interface 120c is connected to the central controller 1530. Communications interface 120c may be connectable to a remote external communication network 1526 and/or a local external communication network 1527 (e.g., the network 112). The remote external communication network 1526 may be connectable to a remote external device 1524. The local external communication network 1527 may be connectable to a local external device 1534.

In one or more embodiments, remote external communication network 1526 is the Internet. The communications interface 120c may use wired communication or wireless communications to connect to the Internet. In one or more embodiments, local external communication network 1527 utilizes one or more communication standards, such as Bluetooth, a consumer infrared protocol, an I/O bus such as a universal serial bus (USB), peripheral component interconnects (PCIs), etc.

In one or more embodiments, remote external device 1524 is one or more computers, for example a cluster of networked computers. In one or more embodiments, remote external device 1524 may be virtual computers, rather than physical computers. In either case, such a remote external device 1524 may be accessible to an appropriately authorized person such as a clinician.

The local external device 1534 represents the external devices 104, which may be a personal computer, mobile phone, tablet, or remote control.

An output device 1529 may take the form of one or more of a visual, audio, and haptic unit. A visual display 1533 may be a Liquid Crystal Display (LCD) or Light Emitting Diode (LED) display. A display driver 1532 receives as an input the characters, symbols, or images intended for display on the display 1533, and converts them to commands that cause the display 1533 to display those characters, symbols, or images.

A display 1533 is configured to visually display characters, symbols, or images in response to commands received from the display driver 1532. For example, the display 1533 may be an eight-segment display, in which case the display driver 1532 converts each character or symbol, such as the figure “0”, to eight logical signals indicating whether the eight respective segments are to be activated to display a particular character or symbol.

In some embodiments, the air circuit 304 is a conduit or a tube constructed and arranged to allow, in use, a flow of air to travel between two components such as RPT device 106 and the patient interface 303. In particular, the air circuit 304 may be in fluid connection with the outlet of the pneumatic block 1506 and the patient interface. The air circuit may be referred to as an air delivery tube. In some cases there may be separate limbs of the circuit for inhalation and exhalation. In other cases a single limb is used.

In some embodiments, the air circuit 304 may comprise one or more heating elements configured to heat air in the air circuit, for example to maintain or raise the temperature of the air. The heating element may be in a form of a heated wire circuit, and may comprise one or more transducers, such as temperature sensors. In one or more embodiments, the heated wire circuit may be helically wound around the axis of the air circuit 304. The heating element may be in communication with a controller such as a central controller 1530.

As illustrated in FIG. 15D, the power supply 1512 may provide electrical power to the input devices 1509, the central controller 1530, the output device 1529, and the pressure generator 1515. The power supply 1512 may also provide electric energy to other components of the RPT device 106 (or the humidifier 305).

In one or more embodiments, an RPT device 106 includes one or more input devices 1509 in the form of buttons, switches or dials to allow a person to interact with the device. The buttons, switches or dials may be physical devices, or software devices accessible via a touch screen. The buttons, switches or dials may, in one or more embodiments, be physically connected to the external housing 1501, or may, in another form, be in wireless communication with a receiver that is in electrical connection to the central controller 1530.

In one or more embodiments, the input device 1509 may be constructed and arranged to allow a person to select a value and/or a menu option.

In one or more embodiments, the central controller 1530 is one or a plurality of processors suitable to control an RPT device 106. The central controller 1530 is shown in FIG. 15C and FIG. 15D. Suitable processors may be any of various commercially available processors. In one or more embodiments, the central controller 1530 is an application-specific integrated circuit. In another form, the central controller 1530 comprises discrete electronic components.

The central controller 1530 may be configured to receive input signal(s) from one or more transducers 1516, one or more input devices 1509, and/or the humidifier 305. The central controller 1530 may be configured to provide output signal(s) to one or more of an output device 1529, a pressure generator 1515, a therapy device controller 1531, a communications interface 120c, and/or the humidifier 305. Furthermore, central controller 1530 can receive information from or transmit information to earbuds 114a-114b.

In some embodiments, the central controller 1530 is configured to implement the one or more methodologies described herein, such as one or more algorithms which may be implemented with processor-control instructions, expressed as computer programs stored in a non-transitory computer readable storage medium, such as memory 118c. In some embodiments, the central controller 1530 may be integrated with an RPT device 106. However, in some embodiments, some methodologies may be performed by a remotely located device. For example, the remotely located device may determine control settings for a ventilator or detect respiratory related events by analysis of stored data such as from any of the sensors described herein.

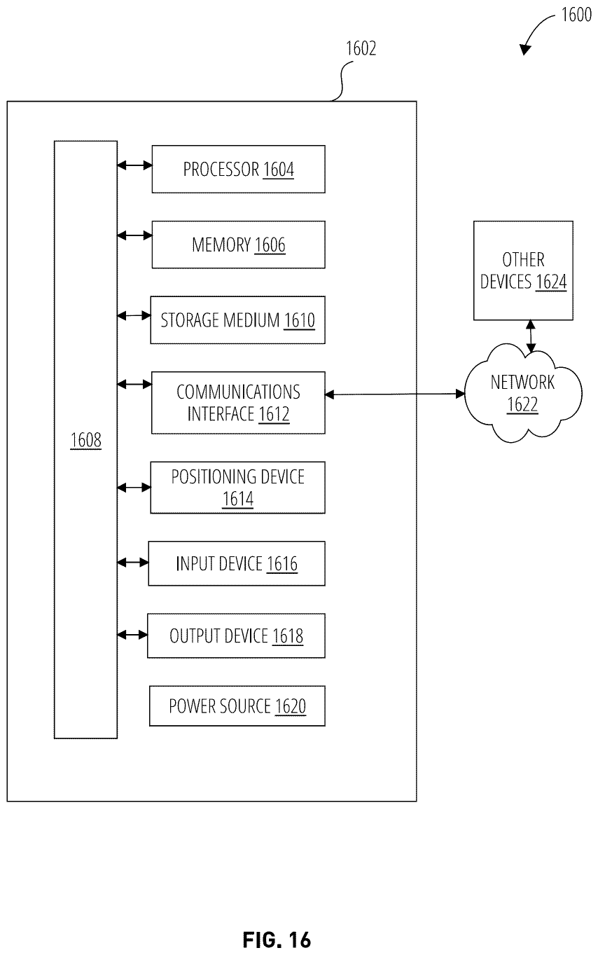

FIG. 16 illustrates an example computing system 1600 suitable for implementing various embodiments as described herein. As shown, the computing system 1600 comprises a computer 1602, which is representative of any type of physical and/or virtualized computing device. Examples of the computer 1602 include, but are not limited to, a server, workstation, laptop, mobile device, smartphone, tablet computer, mainframe, distributed computing system, compute cluster, media device, camera, gaming device, a portable digital assistant (PDA), a system-on-chip (SoC), a pager, a television, a wearable device, a virtual machine (VM), container, or any other device with processing capabilities. In one embodiment, the computer 1602 is representative of some or all of the components of the system 100. More generally, the computing system 1600 is configured to implement all systems, methods, apparatuses, media, and embodiments disclosed herein.

For example, computer 1602 may represent some or all of the components of the earbuds 114a-114b, external devices 104, RPT device 106, mask 108, other wearables 110, and/or device 1204. However, all components of the computer 1602 depicted in FIG. 16 need not be included in the earbuds 114a-114b, external devices 104, RPT device 106, mask 108, other wearables 110, and/or device 1204. Embodiments are not limited in these contexts.

As shown, the computer 1602 includes one or more processors 1604, one or more memories 1606, one or more non-transitory storage media 1610, one or more communications interfaces 1612, one or more positioning devices 1614, one or more input devices 1616, and one or more output devices 1618 communicably coupled via an interconnect 1608. A power source 1620, such as a power supply, battery, or any type of power source may provide power to the computer 1602.

The processor 1604 is representative of any type of processing circuit. For example, the processor 1604 may be a central processing unit (CPU), a microprocessor, a graphics processing unit (GPU), a microcontroller, an application-specific integrated circuit (ASIC), a programmable logic device (PLD), a digital signal processor (DSP), a field programmable gate array (FPGA), a state machine, a controller, gated or transistor logic, a digital signal processor, analog to digital converter, digital to analog converter, and the like.

The memory 1606 is representative of any computer readable medium to store data, code, or other information. The memory 1606 may include volatile memory, such as volatile Random Access Memory (RAM) including a cache area for the temporary storage of data. The memory 1606 may also include non-volatile memory, which can be embedded and/or may be removable. The non-volatile memory can additionally or alternatively include an electrically erasable programmable read-only memory (EEPROM), flash memory or the like. The storage medium 1610 is representative of any type of computer readable medium to store data, code, or other information. Examples of storage media 1610 include solid state drives, hard drives, Redundant Array of Independent Disks (RAID) drives, memory pools, USB storage devices, and the like.

The memory 1606 and storage medium 1610 can store any number and type of computer-executable instructions executed by the processor 1604 to implement the functions of the computer 1602 described herein. For example, the memory 1606 and/or storage medium 1610 may include the therapy application 214, the model 216, the therapies 218, the user profiles 222, and/or the device profiles 224.

The interconnect 1608 is representative of any type of circuitry to connect the components of the computer 1602. For example, the interconnect 1608 can include or represent, a system bus, a universal serial bus (USB) interface, a peripheral component interconnect (PCI), a Peripheral Component Interconnect-enhanced (PCIe), compute express link (CXL) interconnects, Universal Chiplet Interconnect Express (UCIe) interface, PCI-UCIe interconnects, an interface serial peripheral interconnects (SPIs), integrated interconnects (I2Cs), a high-speed interface connecting the processor 1604 to the memory 1606, individual electrical connections among the components, and electrical conductive traces on a motherboard common to some or all of the above-described components of the computer 1602. As discussed herein, the interconnect 1608 may operatively couple various components with one another, or in other words, electrically connects those components, either directly or indirectly—by way of intermediate component(s)—with one another.

The one or more input devices 1616 are representative of any type of input device for receiving input, such as a keypad, keyboard, touchscreen, touchpad, microphone, camera, fingerprint sensor, mouse, joystick, other pointer device, button, soft key, and the like. The one or more output devices 1618 are representative of any type of device for outputting information, such as a monitor, speaker, haptic feedback module, printer, and the like.

The computer 1602 may use the communications interface 1612 to communicate with one or more other devices 1624 via a network 1622. The communications interface 1612 allows the computer 1602 to communicate with and conduct transactions with other devices and systems, such as the other devices 1624. The communications interface 1612 may be a wired and/or a wireless interface. Thus, communications can be conducted, for example, via the wireless communications interface 1612, which can be or include a radio-frequency transceiver, a Bluetooth device, Wi-Fi device, a Near-Field Communication (NFC) device, and other wireless transceivers. In addition, a positioning device 1614 such as a Global Positioning System (GPS) device may be included for navigation and location-related data exchanges, ingoing and/or outgoing. Wi-Fi networks use radio technologies such as IEEE 802.11x (a, b, g, n, ac, ax, etc.) to provide secure, reliable, fast wireless connectivity. A Wi-Fi network connects computers to each other, to the Internet, and to wired networks (which use IEEE 802.3-related media and functions). Communications may also and/or alternatively be conducted via wired connections using the communications interface 1612, e.g., using USB, Ethernet, and other physically connected modes of data transfer. The network 1622 may be any one of, or the combination of, wired and/or wireless networks including without limitation a direct connection, a private network (e.g., an intranet), a public network (e.g., the Internet), a Personal Area Network (PAN), a Local Area Network (LAN), a Wide Area Network (WAN), a wireless network, a cellular network, and other communications networks.

The computer 1602 is configured to use the communications interface 1612 as, for example, a network interface to communicate with one or more other devices on a network such as network 1622. In this regard, the computer 1602 utilizes the wireless communications interface 1612 as an antenna operatively coupled to a transmitter and a receiver (together a “transceiver”) included with the communications interface 1612. The communications interface 1612 is configured to provide signals to and receive signals from the transmitter and receiver, respectively. The signals may include signaling information in accordance with the air interface standard of the applicable cellular system of a wireless network. In this regard, the computer 1602 may be configured to operate with one or more air interface standards, communication protocols, modulation types, and access types. By way of illustration, the computer 1602 may be configured to operate in accordance with any of a number of first, second, third, fourth, fifth-generation communication protocols and/or the like. For example, as a smartphone, the computer 1602 may be configured to operate in accordance with fourth-generation (4G) wireless communication protocols such as Long-Term Evolution (LTE), fifth-generation (5G) wireless communication protocols, Bluetooth Low Energy (BLE) communication protocols such as Bluetooth 5.0, ultra-wideband (UWB) communication protocols, and/or the like. The computer 1602 may also be configured to operate in accordance with non-cellular communication mechanisms, such as via a wireless local area network (WLAN) or other communication/data networks.

The computer 1602 may be under the control of any suitable operating system (not pictured). Example operating systems include, but are not limited to, Linux® operating systems, UNIX®, Windows® operating systems, macOS®, iOS®, Android®, and any other type of operating system.

The computer 1602 as illustrated diagrammatically represents at least one example of a possible implementation, where alternatives, additions, and modifications are possible for performing some or all of the described methods, operations, and functions. Although shown separately, in some embodiments, two or more computers 1602, systems, servers, or illustrated components may be utilized. In some implementations, the functions of one or more systems, servers, or illustrated components may be provided by a single system or server. In some embodiments, the functions of one illustrated system or server may be provided by multiple systems, servers, or computing devices, including those physically located at a central facility, those logically local, and those located as remote with respect to each other.

Aspects of the present disclosure are described herein with reference to flowchart illustrations and/or block diagrams of computer-implemented methods and computing systems according to embodiments of the disclosure. It will be understood that each block of the flowchart illustrations and/or block diagrams, and combinations of blocks in the flowchart illustrations and/or block diagrams, can be implemented by computer readable program instructions that may be provided to a processor of a computer or other programmable data processing apparatus (the term “apparatus” includes systems and computer program products). The processor may execute the computer readable program instructions thereby creating a means for implementing the actions specified in the flowchart illustrations and/or block diagrams. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the actions specified in the flowchart illustrations and/or block diagrams. In particular, the computer readable program instructions may be used to produce a computer-implemented method by executing the instructions to implement the actions specified in the flowchart illustrations and/or block diagrams.

The computer program instructions may also be stored in a computer-readable memory that can direct a computer or other programmable data processing apparatus to function in a particular manner, such that the instructions stored in the computer readable memory produce an article of manufacture including instructions, which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions, which execute on the computer or other programmable apparatus, provide steps for implementing the functions/acts specified in the flowchart and/or block diagram block or blocks. Alternatively, computer program implemented steps or acts may be combined with operator or human implemented steps or acts to carry out an embodiment.

In the flowchart illustrations and/or block diagrams disclosed herein, each block in the flowchart/diagrams may represent a module, segment, or portion of instructions, which comprises one or more executable instructions for implementing the specified logical function(s). In some implementations, the functions noted in the blocks may occur out of the order noted in the Figures. For example, two blocks shown in succession may, in fact, be executed substantially concurrently, or the blocks may sometimes be executed in the reverse order, depending upon the functionality involved.

Computer program instructions are configured to carry out operations of the present disclosure and may be or may incorporate assembler instructions, instruction-set-architecture (ISA) instructions, machine instructions, machine dependent instructions, microcode, firmware instructions, state-setting data, source code, and/or object code written in any combination of one or more programming languages.

An application program may be deployed by providing computer infrastructure operable to perform one or more embodiments disclosed herein by integrating computer readable code into a computing system thereby performing the computer-implemented methods disclosed herein.

Although various computing environments are described above, these are only examples that can be used to incorporate and use one or more embodiments. Many variations are possible.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described to explain the principles of one or more aspects of the disclosure and the practical application, and to enable others of ordinary skill in the art to understand one or more aspects of the disclosure for various embodiments with various modifications as are suited to the particular use contemplated.

What is claimed is:

1. A system, comprising:
a first ear-worn device; and
a second ear-worn device,
the first ear-worn device comprising:
an accelerometer to provide acceleration data; and
a processor operable to execute one or more instructions to cause the processor to:
determine a sleep position of a body wearing the first and second ear-worn devices based on the acceleration data;
determine a pathology of the body;
generate, based on the sleep position of the body and the pathology, a therapy recommendation; and
output an indication of the therapy recommendation.

2. The system of claim 1, wherein the sleep position of the body comprises: (i) a position of a head of the body, and (ii) a position of a torso of the body, the processor operable to execute the one or more instructions to cause the processor to:
receive position data from one or more other devices worn by the body; and
determine, based on the position data from the one or more other devices and the acceleration data from the accelerometer: (i) the position of the head, and (ii) the position of the torso,
wherein the therapy recommendation is further based on the position of the head and the position of the torso.

3. The system of claim 1, the processor operable to execute the one or more instructions to cause the processor to:
generate, based on the position and the pathology, a predicted obstruction of an airway of the body;
generate, based on the predicted obstruction of the airway, an instruction to adjust a parameter of a respiratory therapy system to treat the predicted obstruction; and
transmit the instruction to the respiratory therapy system to cause the respiratory therapy system to adjust the parameter to treat the predicted obstruction.

4. The system of claim 3, the processor operable to execute the one or more instructions to cause the processor to, prior to generating the predicted obstruction:
determine, based on a profile, a count of apneas and a count of hypopneas detected while the body was in the determined position,
wherein the predicted obstruction is further based on the count of apneas and the count of hypopneas.

5. The system of claim 1, the first ear-worn device further comprising two or more microphone arrays, the processor operable to execute the one or more instructions to cause the processor to:
receive an indication of a soundwave detected by the microphone arrays; and
analyze the soundwave to determine an airflow obstruction associated with a respiratory therapy system,
wherein one or more of the sleep position and the therapy recommendation are further based on the determined airflow obstruction.

6. The system of claim 1, the processor operable to execute the one or more instructions to cause the processor to:
detect an apnea or a hypopnea of an airway of the body; and
store, in a profile, an indication of the apnea or the hypopnea associated with the sleep position of the body.

7. The system of claim 1, wherein the pathology comprises sleep apnea, wherein the therapy recommendation comprises changing the sleep position to a different sleep position to treat the sleep apnea, wherein the therapy recommendation is outputted on one or more of: (i) the first and second ear-worn devices, (ii) a smartphone, or (iii) a wearable device.

8. A non-transitory computer-readable storage medium, the computer-readable storage medium including instructions that when executed by a processor of an ear-worn device, cause the processor to:
receive acceleration data from an accelerometer of the ear-worn device;
determine a sleep position of a body wearing the ear-worn device based on the acceleration data;
determine a pathology of the body;
generate, based on the sleep position of the body and the pathology, a therapy recommendation; and
output an indication of the therapy recommendation.

9. The non-transitory computer-readable storage medium of claim 8, wherein the sleep position of the body comprises: (i) a position of a head of the body, and (ii) a position of a torso of the body, wherein the instructions further cause the processor to:
receive position data from one or more other devices worn by the body; and
determine, based on the position data from the one or more other devices and the acceleration data from the accelerometer: (i) the position of the head, and (ii) the position of the torso,
wherein the therapy recommendation is further based on the position of the head and the position of the torso.

10. The non-transitory computer-readable storage medium of claim 8, wherein the instructions further cause the processor to:
generate, based on the position and the pathology, a predicted obstruction of an airway of the body;
generate, based on the predicted obstruction of the airway, an instruction to adjust a parameter of a respiratory therapy system to treat the predicted obstruction; and
transmit the instruction to the respiratory therapy system to cause the respiratory therapy system to adjust the parameter to treat the predicted obstruction.

11. The non-transitory computer-readable storage medium of claim 10, wherein the instructions further cause the processor to, prior to generating the predicted obstruction:
determine, based on a profile, a count of apneas and a count of hypopneas detected while the body was in the determined position,
wherein the predicted obstruction is further based on the count of apneas and the count of hypopneas.

12. The non-transitory computer-readable storage medium of claim 8, wherein the instructions further cause the processor to:
receive an indication of a soundwave detected by two or more microphone arrays of the ear-worn device; and
analyze the soundwave to determine an airflow obstruction associated with a respiratory therapy system,
wherein one or more of the sleep position and the therapy recommendation are further based on the determined airflow obstruction.

13. The non-transitory computer-readable storage medium of claim 8, wherein the instructions further cause the processor to:
detect an apnea or a hypopnea of an airway of the body; and
store, in a profile, an indication of the apnea or the hypopnea associated with the sleep position of the body.

14. The non-transitory computer-readable storage medium of claim 8, wherein the pathology comprises sleep apnea, wherein the therapy recommendation comprises changing the sleep position to a different sleep position to treat the sleep apnea, wherein the therapy recommendation is outputted on one or more of: (i) the ear-worn device, (ii) a smartphone, or (iii) a wearable device.

15. A method, comprising:
receiving, by a processor of an ear-worn device, acceleration data from an accelerometer of the ear-worn device;
determining, by the processor, a position of a body wearing the ear-worn device based on the acceleration data;
determining, by the processor, a pathology of the body;
generating, by the processor based on the position of the body and the pathology, a therapy recommendation; and
outputting, by the processor, an indication of the therapy recommendation.

16. The method of claim 15, wherein the position of the body comprises: (i) a position of a head of the body, and (ii) a position of a torso of the body, the method further comprising:
receiving, by the processor, position data from one or more other devices worn by the body; and
determining, by the processor based on the position data from the one or more other devices and the acceleration data from the accelerometer: (i) the position of the head, and (ii) the position of the torso,
wherein the therapy recommendation is further generated by the processor based on the position of the head and the position of the torso.

17. The method of claim 15, further comprising:
generating, by the processor based on the position and the pathology, a predicted obstruction of an airway of the body;
generating, by the processor based on the predicted obstruction of the airway, an instruction to adjust a parameter of a respiratory therapy system to treat the predicted obstruction; and
transmitting, by the processor, the instruction to the respiratory therapy system to cause the respiratory therapy system to adjust the parameter to treat the predicted obstruction.

18. The method of claim 17, further comprising:
determining, by the processor based on a profile, a count of apneas and a count of hypopneas detected while the body was in the determined position,
wherein the predicted obstruction is further generated by the processor based on the count of apneas and the count of hypopneas.

19. The method of claim 15, further comprising:
receiving, by the processor, an indication of a soundwave detected by two or more microphone arrays of the ear-worn device; and
analyzing, by the processor, the soundwave to determine an airflow obstruction associated with a respiratory therapy system,
wherein one or more of the sleep position and the therapy recommendation are further based on the determined airflow obstruction.

20. The method of claim 15, wherein the therapy recommendation is outputted on one or more of: (i) the ear-worn device, (ii) a smartphone, or (iii) a wearable device, the method further comprising:
detecting, by the processor, an apnea or a hypopnea of an airway of the body; and
storing, by the processor in a profile, an indication of the apnea or the hypopnea associated with the position of the body.
